(19) United States
(12) Patent Application Publication
(10) Pub. No.: US 2025/0345496 A1
(43) Pub. Date: Nov. 13, 2025

(54) A SYSTEM FOR DETERMINING THE MAGNITUDE OF THE ULTRAFILTRATION VOLUME EXPECTED IN A PERITONEAL DIALYSIS TREATMENT

(71) Applicants: Fresenius Medical Care Deutschland GMBH, Bad Homburg (DE); Fresenius Medical Care Holdings, Inc., Waltham, MA (US)

(72) Inventors: Bernd Eberle, Herschweiler-Pettersheim (DE); Paul Chamney, Herts Hertfordshire (GB); Vaibhav Maheshwari, Natick, MA (US); David Jörg, Frankfurt (DE)

(73) Assignees: Fresenius Medical Care Deutschland GMBH, Bad Homburg (DE); Fresenius Medical Care Holdings, Inc., Waltham, MA (US)

(21) Appl. No.: 18/724,817

(22) PCT Filed: Dec. 29, 2022
(86) PCT No.: PCT/EP2022/087988

§ 371 Date: Jun. 27, 2024

Related U.S. Application Data
(60) Provisional application No. 63/295,203, filed on Dec. 30, 2021.

(51) Int. Cl. A61M 1/16; A61M 1/28; G16H 20/40; G16H 40/67

(52) U.S. Cl. CPC A61M 1/1613; A61M 1/28; G16H 20/40; G16H 40/67; A61M 2205/3334; A61M 2205/502

(57) ABSTRACT

The present disclosure relates to a system for predicting the magnitude of ultrafiltration volume expected in a peritoneal dialysis treatment for an individual patient, the system comprising:—a peritoneal treatment machine configured to perform cycles of a peritoneal treatment performed on the patient according to a prescription by controlling at least one actuator and/or valve of the peritoneal treatment machine, the cycles comprising a fill phase, a dwell phase and a drain phase,—a sensor for repeatedly measuring a sensed value during the treatment performed on the patient,—a controller programmed to predict the magnitude of ultrafiltration volume expected during a treatment performed according to a prescription based on a model of ultrafiltration, and to fit parameter values of the model to the individual patient based on the values measured by the sensor, wherein the model of ultrafiltration uses a patient-specific aggregated reflection coefficient as a parameter representing the overall average effect of different pores of the peritoneum and of different solutes present in the peritoneal cavity and the blood plasma on the differential crystalloid osmotic pressure between the peritoneal cavity and blood plasma.

This application is related to co-owned U.S. provisional patent application No. 63/295,211, filed Dec. 30, 2021, which is incorporated by reference as if fully set forth herein.

The described technology relates to a system and a method for predicting the magnitude of the ultrafiltration volume expected in a peritoneal dialysis treatment for an individual patient. The amount of ultrafiltration may in particular be an ultrafiltration volume.

Predicting ultrafiltration volume (UFV) in a peritoneal dialysis (PD) patient is a vitally important capability in the maintenance of the patient's fluid status.

In routine practice, maintaining fluid balance in patients receiving PD treatment remains a major clinical challenge. Currently, if suboptimal fluid status is identified either by patient symptoms or through clinical examination the PD prescription must be revised to bring the patient to a new quasi steady state fluid balance. Even if the desired fluid status target can be specified, finding the ‘right’ prescription to meet this target is a trial and error process.

Knowledge about peritoneal transport characteristics (membrane parameters) are an important step in identifying the right prescription. Typically, membrane parameters differ between patients. Within an individual patient, membrane parameters can change in the longer term due to anatomical changes in the structure of the peritoneal membrane. Short term changes in membrane transport characteristics can also arise due to changes in fluid status or an episode of peritonitis for example. Membrane parameters can be obtained through established methods that have been developed in the past, typically based around the peritoneal equilibration test (PET). However, these methods are cumbersome, costly, time consuming and highly prone to error. More importantly, it is impractical to perform these tests on a basis that is sufficiently frequent that would allow changes in transport characteristics to be tracked in a way that would allow timely treatment intervention. Membrane testing along with trial and error prescriptions consumes valuable clinician time and imposes significant burden to the patient due to the need for frequent visits to the clinic.

Changes in patient's regular lifestyle and/or renal function change the fluid status demanding again the need for prescription revision to compensate. Furthermore, current approaches to provisioning of PD therapy require the patient to maintain stocks of PD fluids of different glucose compositions at home.

Ultrafiltration failure (UFF) or lack of ultrafiltration is a major cause of dropout in PD. While alterations in the anatomical structure of the membrane and changes in hydration status (Dehydration) are in large part responsible in reducing UF capacity, the effects can be mitigated by appropriate changes to the prescription. Unfortunately, in the absence of UF prediction capability, the tendency in clinical practice is to increase the glucose content of the PD fluid. This further increase glucose exposure which is thought to be linked to accelerated membrane damage and unnecessary glucose loading of the patient. Alternative options exist by optimising the dwell period but without methods to determine optimal timing of the drain phase this approach cannot be advanced.

Regarding a PD treatment, fluid status in PD patients is affected by the ultrafiltration volume (UFV). It is the ability to control the UFV (UF prediction) that presents the principle difficulty in making rational changes to the PD prescription. The relationship between the fill volume, PDF (Peritoneal Dialysis Fluid) composition and dwell duration is generally complex. This is compounded by the fact that membrane parameters cannot be assumed constant over a longer period of months due to the reasons highlighted above. Furthermore, transient changes in membrane characteristics may arise at any time due to factors such as infection or changes in hydration status.

There already exist several model approaches for predicting ultrafiltration.

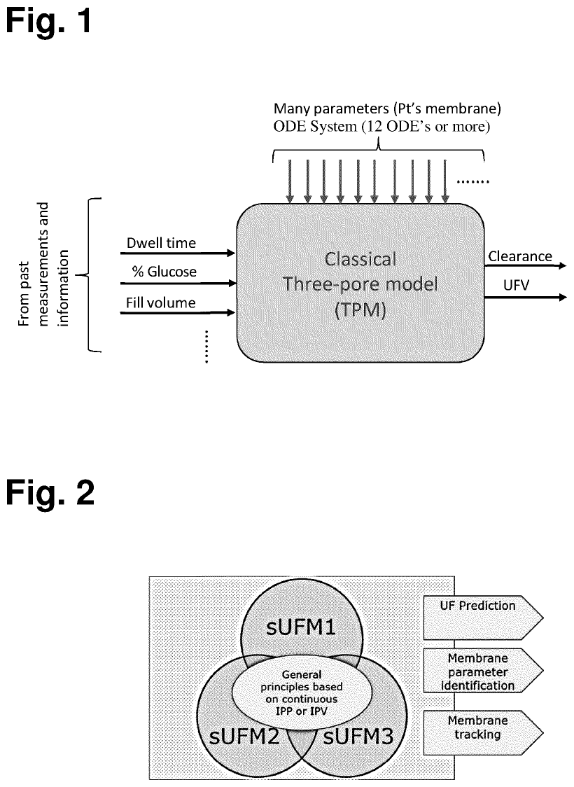

The main approach to predicting ultrafiltration is the Rippe three pore model (TPM). Major advancements in the understanding of hetero pore membranes and transport processes across the peritoneal membrane may be attributed to the work of Bengt Rippe et al. In a publication [Bengt Rippe, Gunnar Stelin, Börje Haraldsson, Computer simulations of peritoneal fluid transport in CAPD, Kidney International, Volume 40, Issue 2, 1991, Pages 315-325, a three-pore model (TPM) is described for the prediction of volume and solute transport across the peritoneal membrane. Three types of membranes are mentioned as Aquaporins, which are only permeable to water, and additional small and large pores. FIG. 1 depicts a high-level representation of this model which serves mainly to call out key input and outputs. While the TPM is based on solid physical principles it comes with relatively high complexity. Along with the colloid effect of proteins in the peritoneal cavity and the plasma, the TPM considers all relevant osmotically active solutes which are transported through the peritoneal membrane and influence transport processes. Many parameters are required for each solute considered, such as effective molecular radius, reflection coefficients and modified Péclet numbers. Consequently, the kinetics of each solute is described by a dedicated differential equation (ODE). This leads to a system of around 12 ODEs dependent on the effects and solutes considered. It is burdensome to obtain all the parameters needed and despite the complexity, UF prediction accuracy still remains poor because of many assumptions [Vonesh E F, Story K O, O'Neill W T. A multinational clinical validation study of PD ADEQUEST 2.0. PD ADEQUEST International Study Group. Perit Dial Int. 1999 November-December;19(6):556-71. PMID: 10641777].

Another approach is the Rippe phenomenological model. Next to the three-pore model, Stelin and Rippe introduced a phenomenological model that describes the nature of the temporal variation of UF during the dwell phase [Gunnar Stelin, Bengt Rippe, A phenomenological interpretation of the variation in dialysate volume with dwell time in CAPD, Kidney International, Volume 38, Issue 3, 1990, Pages 465-472]. The model describes the shape of the dwell curve by a simple mathematical equation and requires only three parameters, offering major simplification. However, because this model cannot really be related to physical quantities, it has limited scope for modification or to consider available information such as solute concentrations, residual volume etc.

Document WO 2018/210904 A1 shows an apparatus for performing peritoneal dialysis, wherein the intraperitoneal pressure is measured during a fill phase in order to determine a function of intraperitoneal pressure to intraperitoneal volume and use this function to generate at least one therapy-related prediction or recommendation, wherein the therapy-related prediction may be ultrafiltration volume.

The described embodiments may provide an improved system and a method for predicting the magnitude of the ultrafiltration volume expected in a peritoneal dialysis treatment for an individual patient.

In a first aspect, the present invention comprises a system for determining at least one parameter regarding a peritoneal dialysis treatment for an individual patient, the system comprising:
a peritoneal treatment machine configured to perform cycles of a peritoneal treatment performed on the patient according to a prescription by controlling at least one actuator and/or valve of the peritoneal treatment machine, the cycles comprising a fill phase, a dwell phase and a drain phase,
a sensor for repeatedly measuring a sensed value during the treatment performed on the patient,
a controller programmed to predict the magnitude of the ultrafiltration volume expected during a treatment performed according to a prescription based on a model of ultrafiltration, and to fit parameter values of the model specific to the individual patient based on the values measured by the sensor,
   wherein the model of ultrafiltration uses a patient-specific aggregated reflection coefficient as a parameter representing the overall average effect of different pores of the peritoneum and of different solutes present in the peritoneal cavity and the blood plasma on the differential crystalloid osmotic pressure between the peritoneal cavity and blood plasma.

By using the aggregated reflection coefficient, the model is much simpler that the usual three-pore model, which uses separate reflection coefficient for all combinations of sizes of pores and solutes, such that it is possible to actually implement the model on a controller and to fit its parameters to a patient. Further, the aggregated reflection coefficient has physical meaning, which allows to use the predictive power of the model for different prescriptions. Further, the model is surprisingly accurate in predicting ultrafiltration.

In an embodiment, the model uses the aggregated reflection coefficient to describe the overall dependency of the differential crystalloid osmotic pressure between the peritoneal cavity and blood plasma on the difference between the osmolarity of the dialysis solution present in the peritoneal cavity and the osmolarity of the blood plasma. This is of particular advantage as the osmolarity of the fresh dialysis solution is known, and that the osmolarity of the blood plasma can be easily measured.

In a second aspect, the present application comprises a system for determining at least one parameter regarding a peritoneal dialysis treatment for an individual patient, in particular a system according to any one of the preceding claims, the system comprising:
a peritoneal treatment machine configured to perform cycles of a peritoneal treatment performed on the patient according to a prescription by controlling at least one actuator and/or valve of the peritoneal treatment machine, the cycles comprising a fill phase, a dwell phase and a drain phase,
a pressure sensor for repeatedly measuring pressure values indicative of the intraperitoneal pressure of the patient during the treatment performed on the patient,
a controller programmed to predict the magnitude of ultrafiltration volume expected during a treatment performed according to a prescription based on a model of ultrafiltration, and to fit parameter values of the model to the individual patient based on the values measured by the sensor, wherein the controller is programmed
to compare values calculated from the sensed pressure values with corresponding values calculated on the basis of the model for fitting the parameter values of the model to the individual patient and
to accept, as an input, a planned prescription, and to output an ultrafiltration volume expected resulting from a treatment performed according to the planned prescription for the individual patient, the ultrafiltration amount calculated on the basis of the model.

The system according to the second aspect will greatly improve the possibility of adapting prescriptions to the needs of a patient as it allows to predict ultrafiltration of a planned prescription before the prescription is used on the patient. Because this prediction is based on a model that is repeatedly updated based on the sensed pressure values, it is much more accurate than prior art methods based e.g. on a PET test.

The systems according to the first and the second aspects form subject matter of the present embodiments independently form each other. Further, the two aspects may be combined. In particular, in the system according to the second aspect, a model as described with respect to the first aspects may be used.

In the following, optional features of the system according to first and/or the second aspect will be described as embodiments.

In an embodiment, the treatment machine may use a pump actuator and/or pump as an actuator and/or valve for performing the cycles of a peritoneal treatment. The pump may be used to pump fresh dialysis fluid to the peritoneal cavity of the patient during a fill phase, and/or to pump used dialysis fluid from the peritoneal cavity of the patient during a drain phase. One example of a pump used in a treatment machine is a diaphragm pump. A pumping chamber of the diaphragm pump may be provided in the form of a disposable connected to a pumping actor of the treatment machine.

In an embodiment, the treatment machine may use gravity to move dialysis fluid to and from the peritoneal cavity. The fluid flow may be controlled using one or more valve actuators and/or valves as an actuator and/or valve of the described embodiments. The valve may for example be provided by a clamp acting on a fluid line to control flow through the fluid line, the clamp forming an actuator of the described embodiments.

In an embodiment, the controller is programmed to receive the overall osmolarity of the dialysis solution instilled into the patient as an input to the model of ultrafiltration. This value is usually known form the manufacturer of the solution.

In an embodiment, the system comprises a user interface configured for inputting a value for the osmolarity. Alternatively or in addition, the controller may be programmed to obtain the value of osmolarity from a data storage. In particular, the peritoneal dialysis machine may be configured to automatically determine the type of solution connected to the machine and, based on this knowledge, determine the osmolarity.

In an embodiment, the controller is programmed to receive the overall osmolarity of the blood plasma as an input to the model of ultrafiltration.

In an embodiment, the system comprises a user interface configured for inputting a value for the osmolarity and/or a measurement device for measuring osmolarity of a blood sample of the patient.

In an embodiment, the osmolarity of the fluid in the peritoneal cavity is a time-dependent state variable of the model. The osmolarity may be described as the number of osmotically active molecules with respect to the volume of the peritoneal cavity.

In an embodiment, the osmolarity of the plasma is considered to be constant in the model.

In a first embodiment, the model describes multiple cycles with fill, dwell and drain phases.

In a second embodiment, the model describes only the dwell phase of single cycles.

In an embodiment, the model uses a patient-specific parameter describing the effective hydraulic conductance to describe the membrane characteristics. The effective hydraulic conductance describes the overall flow through the peritoneal membrane per unit pressure across the wall of the peritoneal membrane. It is specific for an individual patient and dependent on the number of active pores in the peritoneal membrane.

In an embodiment, the model uses patient-specific dissipation coefficient for crystalloid osmotic pressure to describe the membrane characteristics.

In an embodiment, the model of ultrafiltration is based on at least two or at least three parameters of the patient's peritoneal membrane, in particular on hydraulic conductance and/or mass transfer area coefficient and/or the aggregated reflection coefficient of the membrane.

In an embodiment, the model of ultrafiltration is based on a maximum of seven, preferable on a maximum of five or four parameters of the patient's peritoneal membrane.

The parameter values used in the model are preferably updated regularly on the basis of the values measured by the sensor. The parameter values may also be output by the system in order to allow for a monitoring of the state of the membrane.

In an embodiment, the controller is programmed to fit the parameter values of the model at least on the basis of several consecutive values sensed over the course of a single dwell phase and/or sensed over different cycles of a treatment.

In an embodiment, the controller is programmed to determine, on the basis of several consecutive values sensed over the course of a treatment and/or a dwell phase, a curve of the intraperitoneal volume of the patient during the treatment and/or the dwell phase, which is compared to a corresponding curve determined by the model to fit the parameter values of the model.

In an embodiment, the controller is programmed to determine, on the basis of several consecutive values sensed over different cycles of a treatment, a curve of the intraperitoneal volume of the patient during the different cycles of the treatment, which is compared to a corresponding curve determined by the model to fit the parameter values of the model.

By using several consecutive values and/or determining a curve, the parameter values of the model can be determined much more quickly and precisely.

In an embodiment, the sensor is a pressure sensor configured to measure pressure values indicative of the intraperitoneal pressure of the patient.

In an embodiment, the controller is programmed to determine, on the basis of the pressure values, the intraperitoneal volume of the patient.

In an embodiment, the controller establishes a pressure/volume-characteristic of the individual patient in a first cycle and/or a fill and/or drain phase and uses the pressure/volume-characteristic to determine the intraperitoneal volume during a dwell phase.

The determination of the pressure/volume-characteristic and the determination of the intraperitoneal volume from the intraperitoneal pressure can in particular be performed as described in document WO 2018/210904 A1, the content of which is included in the disclosure of the present application in its entirety by reference.

In an embodiment, the sensor is part of the peritoneal treatment machine and/or measures a pressure in an extracorporeal fluid line connected to the peritoneal treatment machine.

In an embodiment, the peritoneal treatment machine is configured to perform consecutive cycles differing in at least one out of fill volume, dwell time and composition of the dialysis solution, wherein the controller is programmed to use sensed values measured during the cycles for fitting the parameter values. Such cycles can be used as calibration cycles for initially fitting the parameters of the model to the patient.

In an embodiment, the controller is programmed to continuously update the parameters of the model when treatments are performed on the patient.

In an embodiment, the controller is programmed to output at least one parameter describing the current state of the peritoneum of the patient. In particular, the parameter may be at least one out of the aggregated reflection coefficient, the hydraulic conductance and/or the mass transfer area coefficient of the membrane. The controller can therefore be used for monitoring the state of the membrane.

In an embodiment, the controller is programmed to accept, as an input, a planned prescription, and to output an ultrafiltration amount resulting from the planned prescription for the individual patient, the ultrafiltration amount calculated on the basis of the model.

In an embodiment, the controller is programmed to allow a caregiver of patient to select a prescription for use in a treatment. The peritoneal treatment machine may be configured to run a prescription selected in this way.

In an embodiment, the controller is part of the peritoneal treatment machine. In particular, the controller may be part of a control unit of the peritoneal treatment machine. The user interfaces described above may equally be part of the peritoneal treatment machine.

In an embodiment, the controller is arranged remotely from the peritoneal treatment machine. For example, the controller may be the processor of a computer, such as a personal computer or a server computer.

The functions described above with respect to the controller may be provided by a computer program stored on a non-volatile storage medium of the peritoneal treatment machine and/or the computer and running on the processor of the peritoneal treatment machine and/or the computer. The computer program may be a program for treatment planning, in particular a program for establishing prescriptions for patients, and may use the model for predicting the amount of ultrafiltration achieved by a particular prescription.

The described embodiments may further include a computer program comprising instructions that will, when executed on a controller, perform the functions of the controller described above or in the following.

In particular, the computer program may be stored on a non-volatile storage any will, when executed on a controller, such as a control unit of a peritoneal device or a processor of a computer such as a personal computer or a server computer, perform the functions of the controller described above or in the following. In particular, the computer program may be configured as described above and in the following.

The described embodiments may further include a method for determining at least one parameter regarding a peritoneal dialysis treatment for an individual patient, the method comprising:
repeatedly measuring a sensed value during a peritoneal treatment performed by a peritoneal treatment machine according to a prescription,
predicting the magnitude of the ultrafiltration expected during a treatment performed according to a prescription based on a model of ultrafiltration, and
fitting parameter values of the model to the individual patient based on the values measured by the sensor,
   wherein the model of ultrafiltration uses a patient-specific aggregated reflection coefficient as a parameter representing the overall average effect of different pores of the peritoneum of the individual patient and different solutes present in the peritoneal cavity and the blood plasma on the differential crystalloid osmotic pressure between the peritoneal cavity and blood plasma.

In an embodiment, the method may perform any one of the steps described above or in the following with respect to the systems of the present invention.

In an embodiment, the method may use any one of the systems of the present invention described above or in the following.

By way of example, specific embodiments of the disclosed systems and methods will now be described, with reference to the accompanying drawings.

The figures show:

FIG. 1 a high-level representation of the Rippe 3-pore model;

FIG. 2 advancements of the PD treatment process through sUFM's;

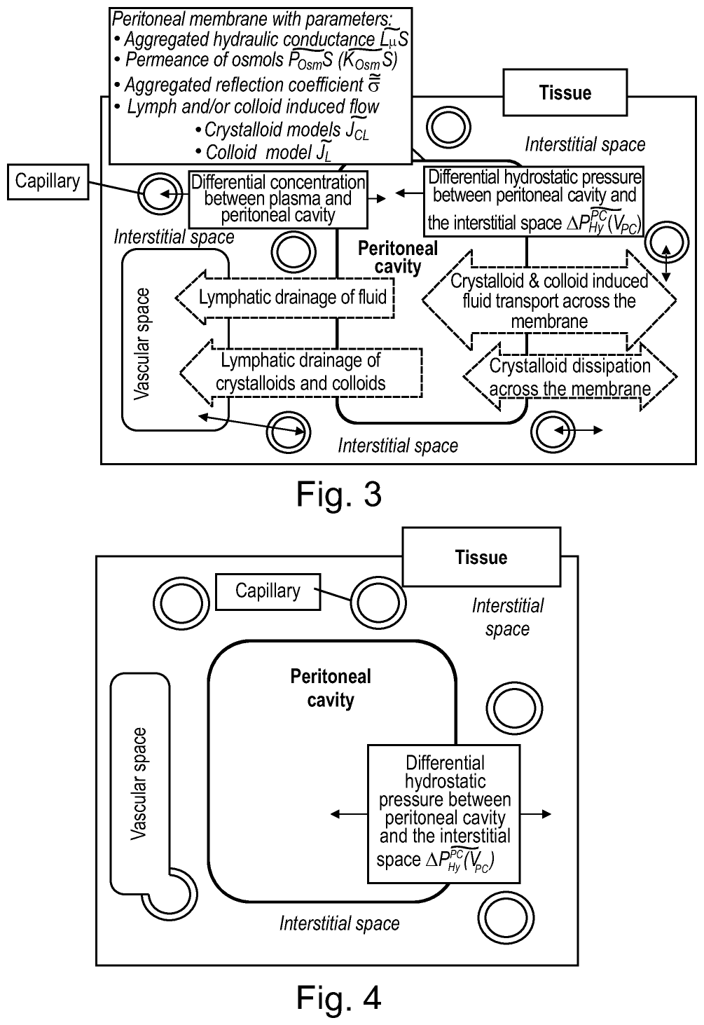

FIG. 3 a model of peritoneal transport kinetics for the purposes of UF prediction

FIG. 4 a schematic representation of the differential hydrostatic pressure across the peritoneal membrane

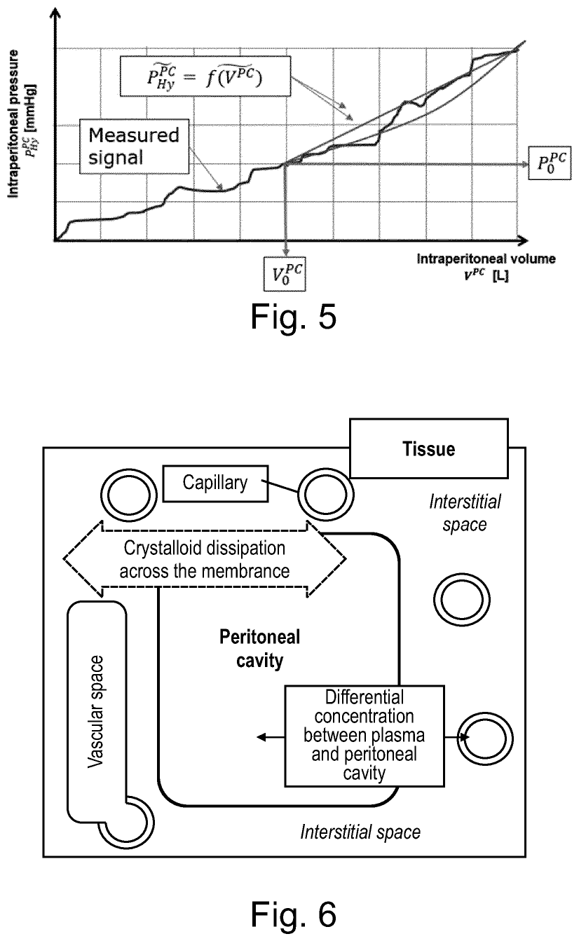

FIG. 5 an example of a ‘Pressure-Volume’ characteristic of the peritoneal cavity

FIG. 6 a schematic representation of the concentration gradients which are generating the osmotic pressures

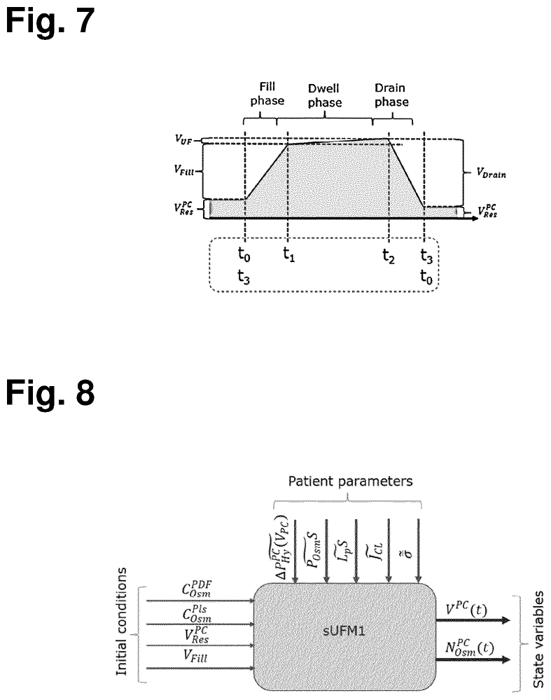

FIG. 7 a parameterisation of the PD cycle

FIG. 8 a schematic representation of the ODE system

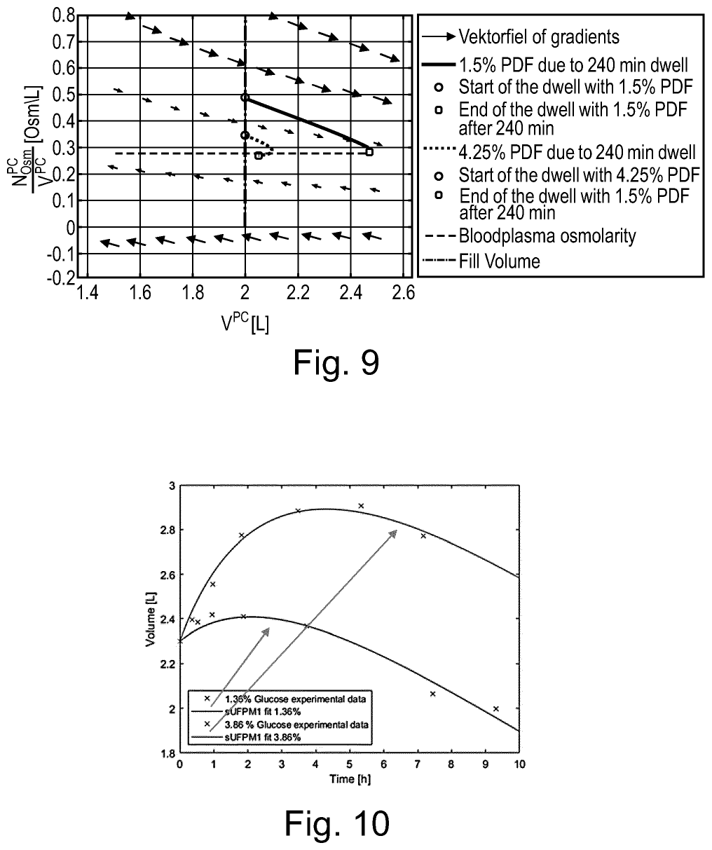

FIG. 9 a vector field of the ODE system: examples for the model gradients at t_1 as a function of osmolarity and peritoneal cavity volume

FIG. 10 raw data of a study with a low and a high glucose PDF and the resulting curve fit by the new model by parameter identification

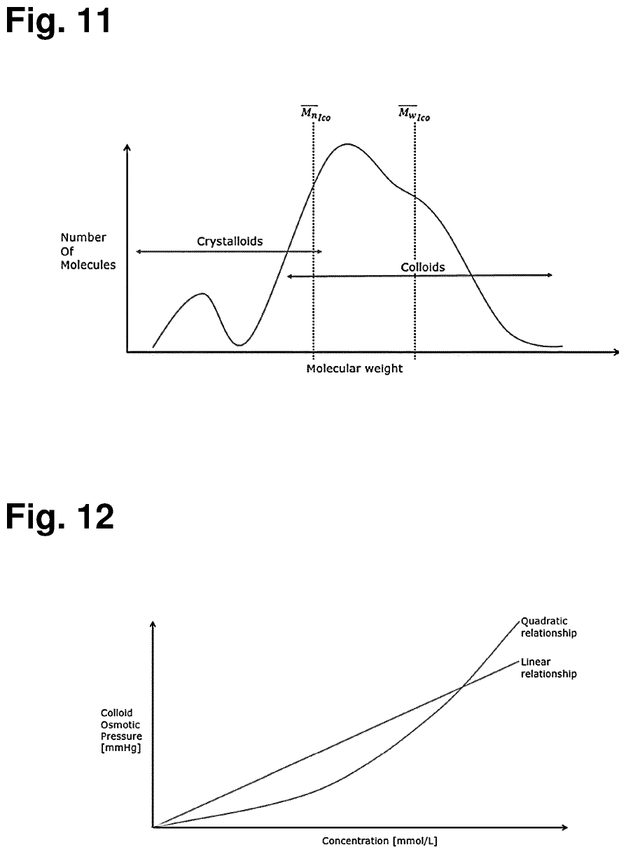

FIG. 11 a schematical representation of the molecular weight distribution of a Polydextrin solution

FIG. 12 a representation of the relationship between colloid concentration and resulting colloid osmotic pressure, somehow like a calibration curve

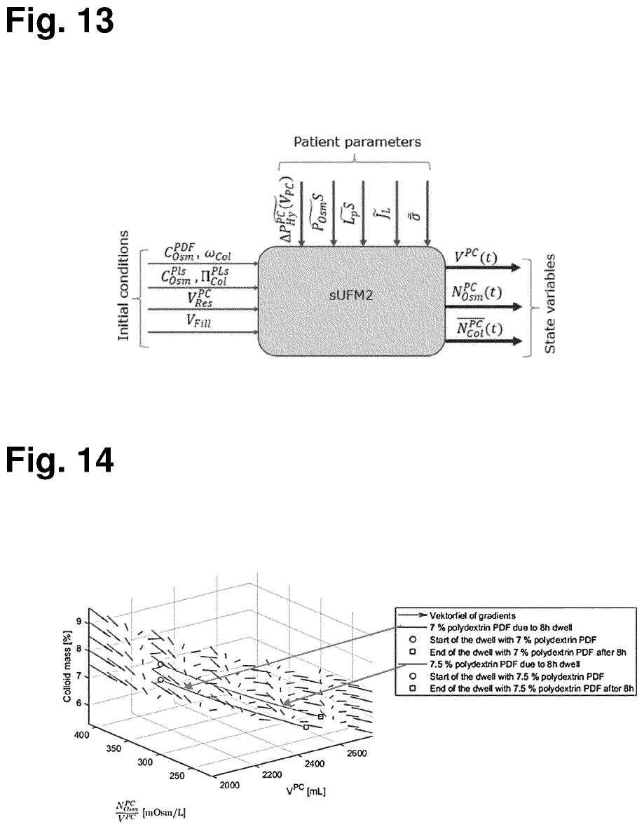

FIG. 13 a schematic representation of the model inputs, outputs and parameters

FIG. 14 a vector field sketch of the sUFM2 ODE system

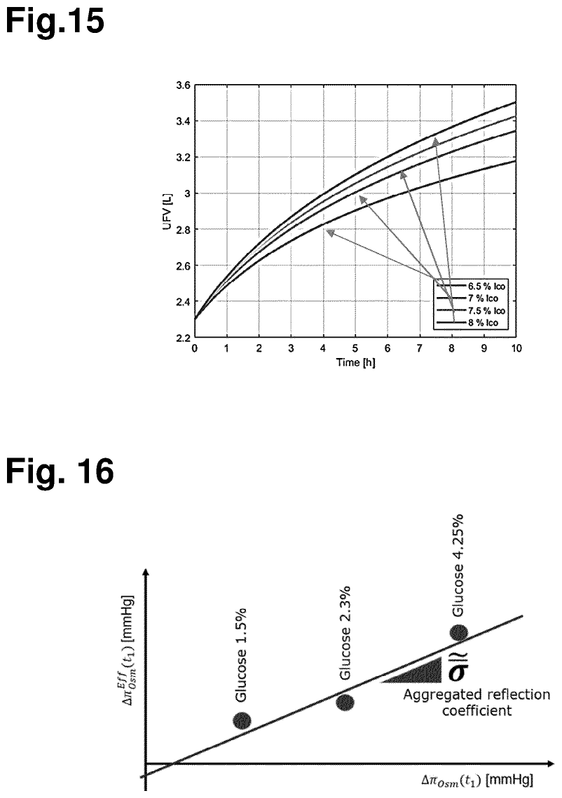

FIG. 15 a simulation of a dwell with a polyglucose solution using the Rippe TPM parameters as an example

FIG. 16 a schematic representation of the relationship of unscaled to scaled osmotic pressure if the membrane is semipermeable

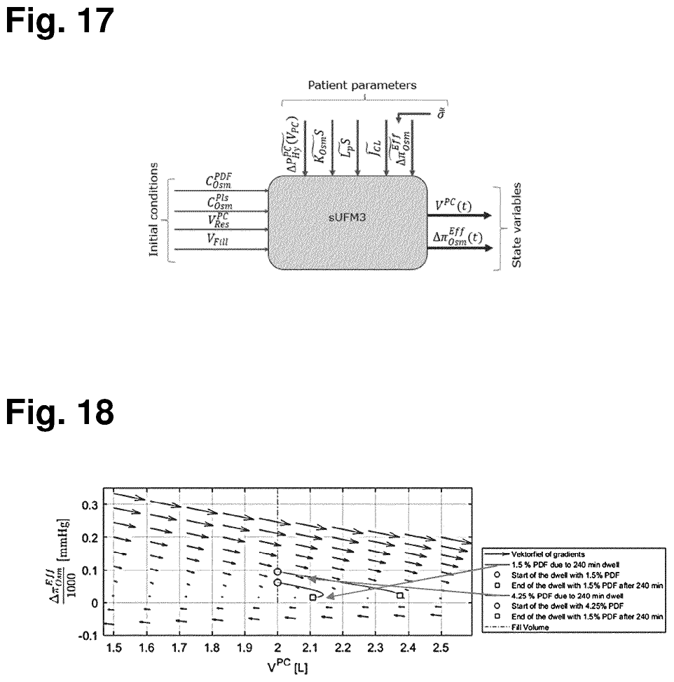

FIG. 17 a schematic representation of the model inputs, outputs and parameters

FIG. 18 a vector field sketch of the sUFM2 ODE system

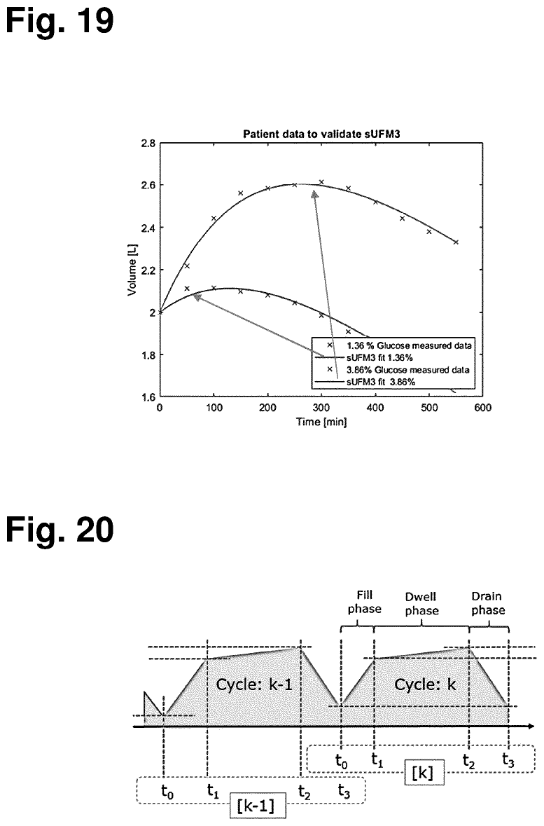

FIG. 19 patient data to validate sUFM3

FIG. 20 consecutive calibration cycles directly followed by each other

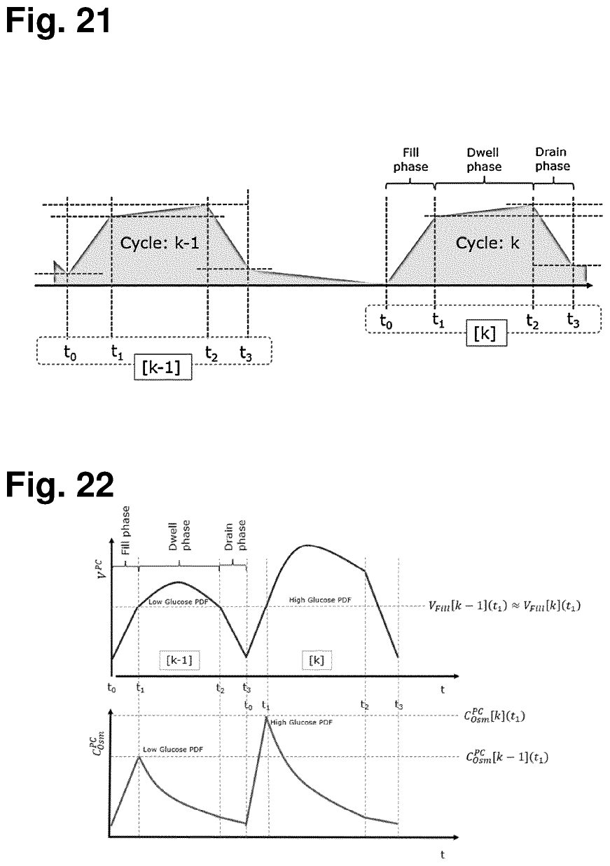

FIG. 21 consecutive calibration cycles with a dry phase in between, which allows the residual volume to be reabsorbed again

FIG. 22 consecutive calibration cycles with different amounts of osmotic-agent (OA)

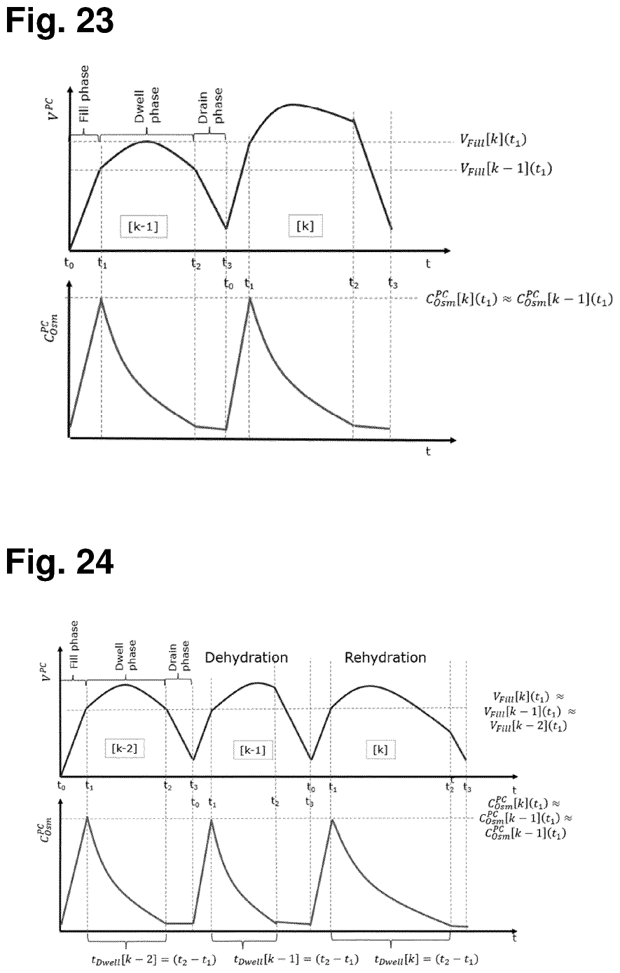

FIG. 23 consecutive calibration cycles with different fill volumes

FIG. 24 consecutive calibration cycles with different dwell times

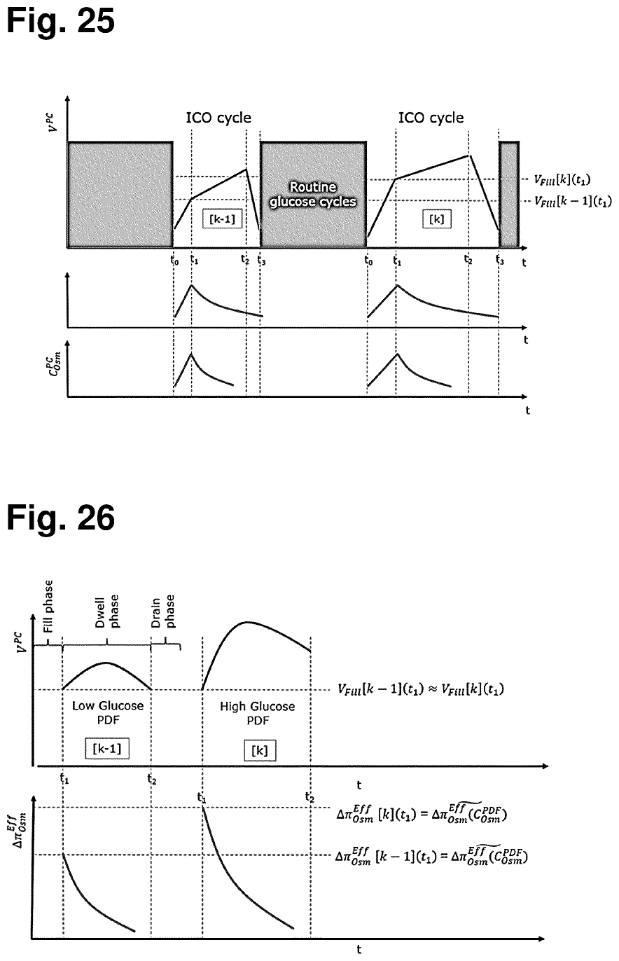

FIG. 25 a design of calibration PD cycles for sUFM2

FIG. 26 a design of calibration PD cycles for sUFM3 and

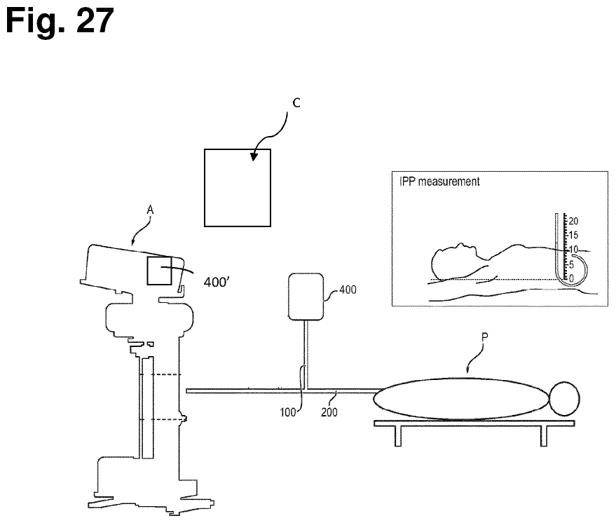

FIG. 27 a first embodiment of a system in accordance with the present disclosure

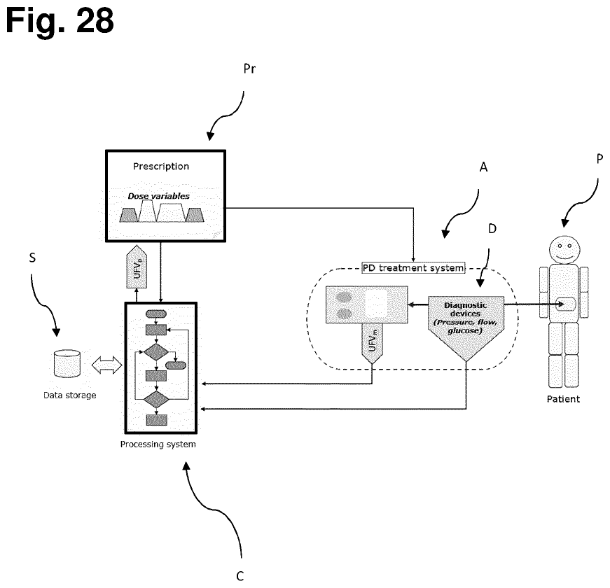

FIG. 28 a second embodiment of a system in accordance with the present disclosure;

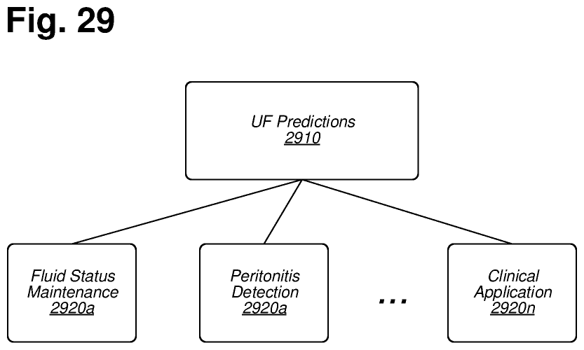

FIG. 29 depicts clinical applications associated with UFV predictions in accordance with the present disclosure;

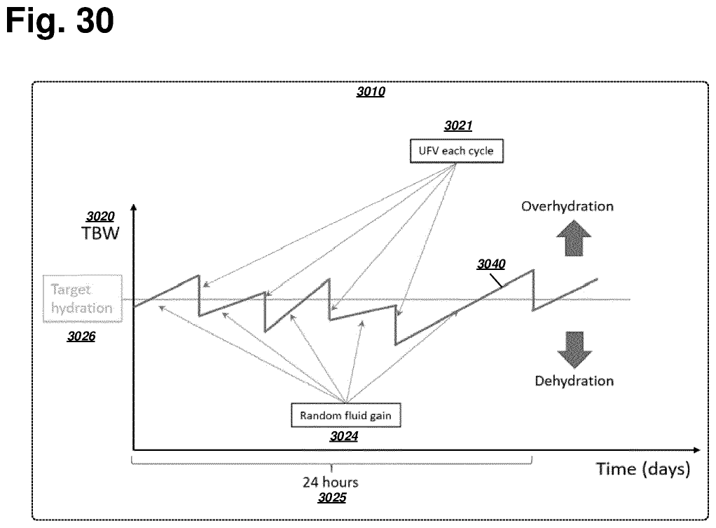

FIG. 30 depicts a graph of variation in total body water (TBW) over a 24-hour period in accordance with the present disclosure;

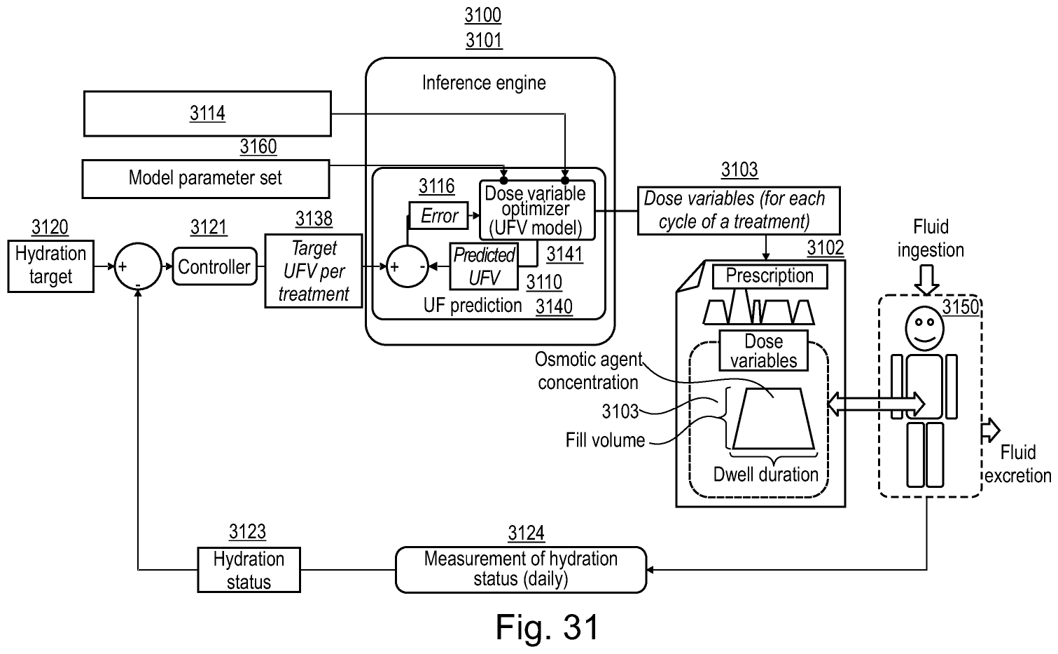

FIG. 31 illustrates an exemplary operating environment of patient hydration maintenance in accordance with the present disclosure; and

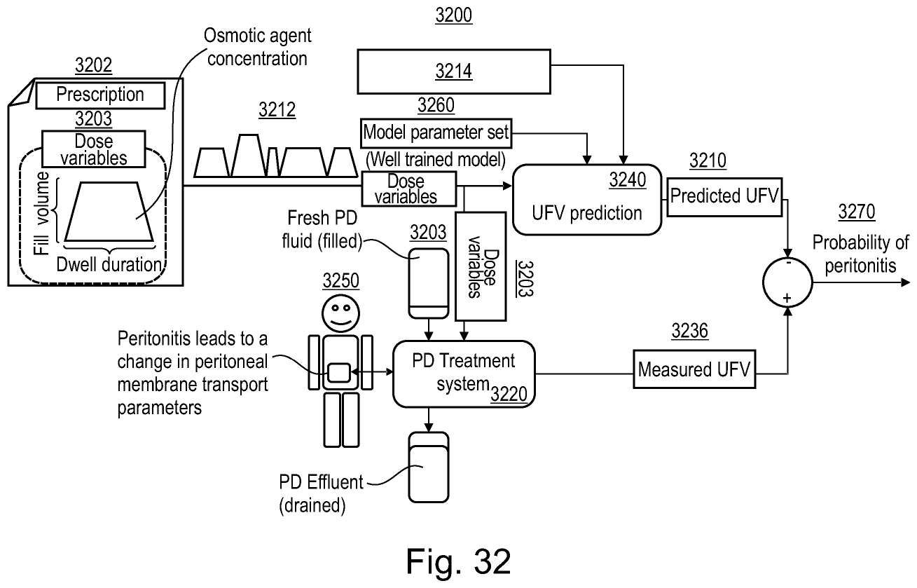

FIG. 32 illustrates an exemplary operating environment of peritonitis determination or prediction in accordance with the present disclosure

Predicting ultrafiltration volume (UFV) in a peritoneal dialysis (PD) patient is a vitally important capability in the maintenance of the patient's fluid status.

In an embodiment, a first principle model has been developed to predict UFV based on two key characteristics (parameters) of the patient's peritoneal membrane, namely hydraulic conductance and mass transfer area coefficient. The model involves a few ordinary differential equations and represents a major simplification over existing models that have been developed over recent decades. The model offers greatly improved accuracy which is afforded by regular update of the patient's peritoneal membrane parameters. This affords the capability to adapt the model to long-term and short-term changes in the patient's peritoneal membrane parameters.

In an embodiment, an update of the patient parameters is achieved using continuous measurements of intraperitoneal pressure (IPP) during all phases of the PD treatment cycle. This provides the primary source of data from which membrane parameters can then be derived. Continuous IPP measurements leverages PD systems with built-in pressure transducers thus providing the basis to fully automate the process of identifying membrane parameters at every PD cycle.

### 1 Basic Layout of the System

An embodiment of a system is shown in FIG. 27. The system comprises a peritoneal treatment machine A configured to perform a peritoneal treatment on a patient P. For this purpose, a fluid line 200 connected to actuators and/or valves of the treatment machine A is provided for alternately filling and draining the peritoneal cavity of the patient P with dialysis solution.

The peritoneal treatment machine comprises a control unit configured to control the actuators and/or valves to perform the treatment on the patient according to a prescription. A prescription defines at least the dwell times. Further, the prescription may define fill and/or drain volumes. The prescription may be stored on a patient card, which is read by the control unit to adapt the treatment to the patient. The prescription may equally be provided by a server computer connected to the peritoneal treatment machine via the internet.

Preferably, the peritoneal treatment machine is an automated peritoneal treatment machine, which is configured to perform the treatment, once initiated, automatically and without human intervention.

In an embodiment, the fresh dialysis solution is provided in solution bags. The solution bags are connected via at least one fluid line with the patient. The peritoneal treatment machine will control the flow from the solution bags to the patient via its actuators and/or valves.

In a first embodiment, the peritoneal treatment machine comprises, as an actuator, at least one pump for pumping fresh dialysis solution to the patient and draining spend dialysis solution from the patient. In a second embodiment, the peritoneal treatment machine may use gravity for filling and draining, and may control the treatment by stopping and starting flow through fluid lines connecting the fluid bags with the patient, for example by valves. Such valves may be provided as clamps acting on the fluid lines.

The system further comprises a sensor 400, 400′ for sensing a parameter relating to the patient and/or the treatment. In the embodiment, the sensor measures a pressure indicative of intraperitoneal pressure. In the embodiment, the sensor 400, 400′ measure a pressure in a fluid line that is in fluid communication with the peritoneal cavity of the patient P. In order to determine the intraperitoneal pressure from the pressure measured by the sensor, it might be necessary to account for static pressure due to the height difference. In the embodiment, the pressure sensor is attached to a measurement line 100 that is in fluid connection with the patient line 200. Other configurations of the sensor can also be used.

In the embodiment shown, the sensor 400 is provided in a measurement device provided separately from the peritoneal treatment machine. However, preferably, the sensor is part of the peritoneal treatment machine. This is indicated in FIG. 27 by the reference number 400′.

The system further comprises a controller C programmed to predict the magnitude of the ultrafiltration volume expected during a prescription based on a model of ultrafiltration, and to fit parameter values of the model to the individual patient P based on the values measured by the sensor 400, 400′. The functionality of the controller C is described above and in more detail in the following. In particular, at least one of the models described in the following may be used by the controller for predicting ultrafiltration. In particular, the controller may be programmed to use one of the models. Further, it may have the further functions described in the following and above.

In an embodiment, the controller C is part of the peritoneal treatment machine A. In particular, it may be part of a control unit of the peritoneal treatment machine.

In another embodiment, the controller C is provided separately from the treatment machine A and may for example be part of a computer. In this case, the computer may have a computer program stored, the computer program implementing the functionality of the controller. The computer program may be a program for generating, planning and/or evaluating prescriptions.

A second embodiment of a system for controlling UF in a PD patient using of a model of ultrafiltation sUFM is shown in FIG. 28. The model predicts the magnitude of the ultrafiltration volume UFVₚ expected during a future treatment performed according to a first prescription. It may use, as an input, the measured UF volume UFMm for at least one past treatment performed according to a second prescription.

The system depicted in FIG. 28 may have the same basic components as the system shown in FIG. 27. Therefore, the same reference signs are used for those components in FIGS. 27 and 28, and the above description of the system of FIG. 27 equally applies to the system of FIG. 28.

The system of FIG. 28 is described in further detail in the following table.

| Component | Description |
|---|---|
| Prescription | The prescription specifies the ‘dose variables’ for a |
| Pr | given treatment. The dose variables may comprise: |
| | fill volume, |
| | glucose composition |
| | Dwell duration |
| | Number of cycles |
| | The prescription may be generated by a prescription |
| | generation tool, e.g. in the form of a computer program |
| | used to generate prescription. The prescription may be |
| | transferred to the PD Treatment System and used by the |
| | PD Treatment System to control a treament. |
| PD Treatment | The system for performing peritoneal dialysis (fill the |
| system A | patient's peritoneal cavity with PD dialysing fluid and |
| | drain the effluent after it has dwelled in the cavity for |
| | several hours), also referred to as peritoneal treatment |
| | machine in the present description. |
| | The treatment system may be either CAPD, APD with |
| | fluid exchange performed by gravity or mechanical |
| | pump. |
| | The treatment system is equipped with diagnostic |
| | sensors that provide the source information for the |
| | processing system. |
| | Additionally, the measured UF volume, UFMₘ is an |
| | input to the processing system. |
| Diagnostic | Additional sensors integrated with a PD treatment system |
| devices D | that provide dynamic information during each PD cycle. |
| | Diagnostic sensors may sense one or more of the |
| | following values: |
| | Flow rate during fill & drain as well as UFVₘ |
| | Intraperitoneal Pressure (IPP), monitored during the |
| | fill, dwell and drain phases of a PD cycle |
| | Glucose composition of PD effluent, monitored at |
| | intervals during the dwell |
| Processing | The processing system (also referred to as controller |
| system C | in the present description) encapsulates the sUFMs that |
| | require as input the dose variables (initial conditions) |
| | and corresponding diagnostic sensor data and UFVₘ |
| | information. The processing system performs the |
| | following functions: |
| | Determination of patient peritoneal transport |
| | parameters by solving the system equations represented |
| | by the sUFMs using past treatment data |
| | Prediction of the UF volume (UFVₚ) for future cycles |
| | and treatments based on a sUFM. This requires as input |
| | the prescription (Dose variables → initial conditions) |
| | and the most recent patient peritoneal transport |
| | parameters |
| Storage | Enables the processing system to be fed with historical |
| system S | data comprising one or more of |
| | Prescriptions (dose variables) |
| | Treatment data |
| | Patient transport parameters |

### 2 Introduction

The method described herein for UF prediction by first principles overcomes several limitations in current approaches, offering the potential for greatly improved accuracy. Whereas current methods attempt to predict the temporal variation of intra peritoneal volume (IPV), relying on several assumptions in the process, the method described here takes an estimate of IPV based on direct measurement of intraperitoneal pressure (IPP). Additionally, the method presented is general to any osmotic agent present in the peritoneal dialysis fluid, provided its crystalloid and colloid properties can be determined.

This report is structured as follows: In Section 2, a common set of physical principles upon which the models are based are introduced. Three variants of the simplified ultrafiltration models (sUFM1, sUFM2, sUFM3) and their scope are presented in Sections 3-5. The methods to obtain specific membrane parameters are outlined in Section 6 and the calibration of the models are discussed in Section 7. Section 8 relates to a continuous real time calibration. An overview of all the simplified UF models is provided in Section 9, while Section 10 highlights possible further adaptations of the models such as the inclusion of addition fluid pools/compartments.

### 3 Basic Physical Principles and Assumptions

### 3.1 General Principles Common to the Simplified UF Prediction Models

The three simplified ultrafiltration models sUFM1, sUFM2 and sUFM3 introduced in Sections 3-5 are proposed for the purpose of UF prediction. Typically, each sUFM reduces the classical TPM to several ODEs and some parameters of physical meaning. Although the sUFMs are simpler and lend themselves very well to practical routine application, they also offer significantly improved prediction accuracy. The accuracy improvement is achieved by continuous measurement of the Intraperitoneal Pressure (IPP) or Intraperitoneal Volume (IPV) as an input. IPP in particular can be measured using available hardware particularly in APD cycler platforms. By capturing the actual variation of IPP (and hence IPV) many of the assumptions necessary in predicting dwell behaviour can be removed. This enables the deployment of automated methods for identification of peritoneal membrane parameters. Consequently, membrane parameters can be tracked continually at the resolution of a single cycle. These advancements in the control of the PD treatment process, possible through the application of the sUFMs, are summarised in FIG. 2.

A number of principles common to all sUFM's proposed and the systematic reduction from the TPM are described in the following sections. Also, how the model deals with colloids in the peritoneal dialysis fluid (PDF) and the usage of osmolarity instead of concentration as a significant simplification would be discussed. These relate mainly to the handling of IPP or IPV as an input and the simplification of the fluid transport kinetics governed by the osmotic drivers.

### 3.2 Transport Processes

The general transport processes considered in our models are very similar to those of the Rippe Three Pore Model (TPM)). The main transport phenomena considered in the sUFMs are illustrated in FIG. 3.

The models consider the interstitial space, the vascular space and the peritoneal cavity. Fluid transport due to lymphatic drainage, differential hydrostatic pressure and concentration gradient-induced fluid flows are indicated by arrows in the middle of the FIG. 3. Additionally, solute transport by diffusion, convection and lymph drainage are taken into account, depending on whether the specific model approach considers colloids and/or crystalloids.

### 3.3 Differential Hydrostatic Pressure

To introduce the models, we need to specify the relationship between intraperitoneal pressure (IPP) and intraperitoneal volume (IPV), which is the major input to determine the model parameters in a frequent way and with sufficient accuracy.

| Term | Description |
|---|---|
| | Differential hydrostatic pressure through the peritoneal membrane as function of the |
| | intraperitoneal volume |

FIG. 4 is a schematic representation of the differential hydrostatic pressure across the peritoneal membrane. The hydrostatic pressure which is present in the peritoneal cavity due to the cavity volume would push the fluid through the peritoneal membrane and out of the peritoneum to equilibrate with the interstitial pressure. The volume and the pressure need to be tracked during in- and outflow together with the corresponding intraperitoneal pressure to capture the pressure-/volume (P/V) characteristics. The pressure in the interstitial space is known to be influenced by the patient's considered to be a function of the hydration status. Generally there is less variation in interstitial pressure especially when the patient is maintained euvolemic by treatment.

### 3.3.1 P/V Characteristic of the Peritoneal Cavity

When PD fluid is introduced into the peritoneal cavity an intraperitoneal pressure (IPP) is developed for a given volume of PD fluid instilled—the so-called pressure-/volume (P/V) characteristic of the peritoneal cavity. This characteristic is denoted by [symbol] , see FIG. 5.

The hydrostatic peritoneal pressure difference across the membrane as a function of peritoneal cavity volume has been described for a given patient posture by [Vicente Pérez Díaz, Sandra Sanz Ballesteros, Esther Hernández García, Elena Descalzo Casado, Irene Herguedas Callejo, Cristina Ferrer Perales, Intraperitoneal pressure in peritoneal dialysis, Nefrología (English Edition), Volume 37, Issue 6, 2017, Pages 579-586, ISSN 2013-2514, https://doi.org/10.1016/j.nefroe.2017.11.002]. The drawback of this approach is that the relationship applies to a patient population rather than the individual patient. Differences in hydrostatic offsets and the compliance of the peritoneal cavity is likely to vary between patients, especially with different age and body weight (parameters

$${P}_{0}^{PC}$$

and γ, respectively).

$$= {P}_{0}^{PC} + γ \cdot ( {V}^{PC} ( t ) - {V}_{0}^{PC} ) \qquad Equation 1$$

| Term | Description |
|---|---|
| [symbol] | Patient specific relationship between the volume in |
| | the peritoneal cavity and the resulting |
| | intraperitoneal pressure |
| ${P}_{Hy}^{PC}$ | Pressure in the intraperitoneal cavity [mmHg] |
| ${P}_{0}^{PC}$ | Pressure at the volume V₀ |
| V^{PC} | Volume in the intraperitoneal cavity [L] |
| ${V}_{0}^{PC}$ | Volume at the pressure P₀ |
| | Hydrostatic pressure inside the peritoneal cavity as a function of the intraperitoneal volume |

With the proposed membrane test method including measurement of intraperitoneal pressure (IPP) during fill and drain we can get a patient specific characteristic how the pressure in the cavity is scaled. See WO2018/210904A1

$$= f ( {P}_{Meas}^{PC} ( t ) , {V}_{Meas}^{PC} ( t ) ) \qquad Equation 2$$

| Term | Description |
|---|---|
| | Patient specific relationship between the volume in the peritoneal cavity and the resulting |
| | intraperitoneal pressure |
| ${P}_{Meas}^{PC} ( t )$ | Quasi-continuous measurement of the intraperitoneal pressure during fill and drain (dwell) |
| ${V}_{Meas}^{PC}$ | Quasi-continuous measurement of the intraperitoneal volume during fill and drain |

Therefore, it is proposed that

is determined for the individual patient during fill, dwell and/or drain phase by acquisition of V^{PC} and

$${P}_{Hy}^{PC}$$

data, for example via an APD cycler.

$$f ( {P}_{Meas}^{PC} ( t ) , {V}_{Meas}^{PC} ( t ) )$$

then becomes the fit of a suitable function that describes the relationship between V^{PC} and P^{PC} over the practical range of interest. This could be a polynomial of any degree fitted to the measured data by for example a least squares minimization process to determine the coefficients of the describing patient specific equation.

### 3.3.2 Differential Hydrostatic Pressure Across the Peritoneal Membrane

In later research it has been demonstrated that interstitial pressure,

$${P}_{HS}^{Int}$$

is in fact the relevant pressure outside the peritoneal cavity [Zakaria E R, Lofthouse J, Flessner M F. Effect of intraperitoneal pressures on tissue water of the abdominal muscle. Am J Physiol Renal Physiol. 2000 June;278(6):F875-85. doi: 10.1152/ajprenal.2000.278.6.F875. PMID: 10836975]. The interstitial pressure is scaled by the hydration status of the patient. For the purposes of the current simplified UF prediction model a more general form of

may be considered:

$$= - {P}_{HS}^{Int} \qquad Equation 3$$

| Term | Description |
|---|---|
| ${P}_{HS}^{Int}$ | Patient-specific interstitial pressure depending on the hydration status of the patient |

In a subject without renal failure,

$${P}_{HS}^{Int}$$

is typically the range of −3 mmHg. However renal failure leading to fluid overload and free fluid flow across the interstitial matrix will cause

$${P}_{H S}^{Int}$$

to rise a few mmHg above zero.

In a first evaluation of the models, the relationship of pressure and volume from [Twardowski Z J, Prowant B F, Nolph K D, Martinez A J, Lampton L M. High volume, low frequency continuous ambulatory peritoneal dialysis. Kidney Int. 1983 January;23(1):64-70. doi: 10.1038/ki.1983.12. PMID: 6834695.]. was used. For practical applications, this relationship should be determined as patient-specific characteristics and should be measured continuously over every fill and drain phase (dwell).

### 3.4 Osmotic Pressures

In addition to the hydrostatic pressure, osmotic pressures are driving fluid flow across the peritoneal membrane. In the case of a semipermeable membrane like in the peritoneum, there is a transport of substances through the membrane till the equilibrium is reached. The effective osmotic pressures are dependent on the concentration gradient through the membrane and also the membrane characteristics. FIG. 6 is a schematic representation of the concentration gradients which are generating the osmotic pressures.

### 3.4.1 Differential Osmotic Pressure Due to Crystalloid Osmotic Agents

The approach of [Bengt Rippe, Gunnar Stelin, Börje Haraldsson, Computer simulations of peritoneal fluid transport in CAPD, Kidney International, Volume 40, Issue 2, 1991, Pages 315-325, ISSN 0085-2538, https://doi.org/10.1038/ki.1991.216. (https://www.sciencedirect.com/science/article/pii/S0085253815573067)] determines the overall crystalloid osmotic pressure from all relevant solutes (crystalloids). Knowledge of the value of reflection coefficients, σ_{i,m}, is required for each crystalloid i when presented to a given pore m of known size. This demands the application of pore theory as well as an estimate of the effective radius of a given solute and pore.

The established expression for the differential crystalloid osmotic pressure between the peritoneal cavity and blood plasma is given by

$$Δ {π}^{Eff} = {\sum }_{m} {\sum }_{i} {σ}_{i , m} \cdot R \cdot T \cdot ( {C}_{i}^{P C} - {C}_{i}^{P l s} ) \qquad Equation 4$$

where Δπ^{Eff} is the effective differential osmotic pressure across the peritoneal membrane and

| Term | Description | Units |
|---|---|---|
| ${C}_{i}^{Pls}$ | Concentration of the i-th solute in the blood plasma | mosmol/L |
| ${C}_{i}^{PC}$ | Concentration of the i-th solute in the peritoneal cavity fluid | mosmol/L |
| R | Gas constant | kg·m²·S⁻²·mol⁻¹·K⁻¹ |
| T | Temperature | K |
| σ_{i,m} | Reflection coefficient for the i-th solute | |
| | and the m-th pore size | |
| Δπ^{Eff} | Effective crystalloid osmotic pressure | mmHg |
| | which drives a fluid flow through the | |
| | peritoneal membrane | |

$${C}_{i}^{P C} and {C}_{i}^{P l s}$$

are the concentrations of the i^{th} solute in the peritoneal cavity and the plasma respectively.

### 3.4.2 Aggregate Reflection Coefficient

In order to obtain a simplified expression for the effective differential crystalloid osmotic pressure, we introduce an aggregate reflection coefficient \overline{\tilde{σ}}, which represents the overall average effect of different types of pores such as aquaporins, small pores and large pores, the relative pore densities and the solutes present in the peritoneal cavity. The aggregate reflection coefficient is specific to an individual patient and is assumed to be constant on the scale of weeks. Thus, an approximation of Eq. (4) may be derived by the approximation

$${\sum }_{m} {σ}_{i , m} \approx {σ}^{\tilde{\_}} \qquad Equation 5$$

This approximation yields a simplified expression for the differential crystalloid osmotic pressure,

$$\begin{matrix}Δ {π}_{O s m}^{Eff} = {\sum }_{m} {\sum }_{i} {σ}_{i , m} \cdot RT \cdot ( {C}_{i}^{P C} - {C}_{i}^{P l s} ) \\ \approx RT \cdot {σ}^{\tilde{\_}} {\sum }_{i} ( {C}_{i}^{P C} - {C}_{i}^{P l s} ) \\ = {σ}^{\tilde{\_}} \cdot RT \cdot ( {C}_{O s m}^{P C} - {C}_{O s m}^{P l s} )\end{matrix} \qquad Equation 6$$

$$where$$

$${C}_{O s m}^{P C} = {\sum }_{i} {C}_{i}^{P C} and {C}_{O s m}^{P l s} = {\sum }_{i} {C}_{i}^{P l s} .$$

The main simplification is that the concentrations of the solutes are merged together as osmolarities

$${C}_{Osm}^{P C} and {C}_{Osm}^{P l s} ,$$

which are easy to measure by an Osmomat.

| Term | Description | Units |
|---|---|---|
| [symbol] | Aggregated | Dimensionless |
| | reflection | |
| | coefficient | |
| | (all solutes | |
| | considered) | |
| | dependent | |
| | on the | |
| | active pores | |
| | in the | |
| | individual | |
| | patient | |
| RT | Product of | kg·m²·S⁻²·mol⁻¹ |
| | universal | |
| | Gas | |
| | constant | |
| | and | |
| | temperature | |
| ${C}_{Osm}^{Pls}$ | Osmolarity of the blood | mosmol/L |
| | plasma | |
| ${C}_{Osm}^{PC}$ | Osmolarity of the | mosmol/L |
| | peritoneal | |
| | cavity | |
| | volume | |
| ${Δπ}_{Osm}^{Eff}$ | Effective crystalloid | mmHg |
| | osmotic | |
| | pressure | |
| | which | |
| | drives a | |
| | fluid flow | |
| | through the | |
| | peritoneal | |
| | membrane | |
| | only | |
| | considering | |
| | osmoles | |

The number of osmoles is dominated by crystalloids such as glucose especially for glucose-based PD solutions, with smaller contributions from Urea, Creatinine and physiological electrolytes. Any colloids present make a negligible contribution to the number of osmoles.

The sUFM approach by contrast, makes no assumptions regarding reflection coefficients of individual crystalloid pore size pairs or the relative proportions of different pores in the peritoneal membrane. Instead, sUFM considers the net contribution of all crystalloids expressed as osmoles rather than individual crystalloids.

$${C}_{O s m}^{P C} = \frac{{N}_{O s m}^{P C}}{{V}^{P C}} \qquad Equation 8$$

Under this condition we introduce the expression for the differential crystalloid osmotic pressure between the peritoneal cavity and the blood capillaries is given by

$$Δ {π}_{O s m}^{Eff} = {σ}^{\tilde{\_}} \cdot RT \cdot ( {C}_{O s m}^{P C} - {C}_{O s m}^{P l s} ) = {σ}^{\tilde{\_}} \cdot RT \cdot ( \frac{{N}_{O s m}^{P C}}{{V}^{P C}} - {C}_{O s m}^{P l s} ) \qquad Equation 9$$

| Term | Description | Units |
|---|---|---|
| ${N}_{Osm}^{PC}$ | Amount of osmotically active molecules in the peritoneal cavity | mosmol |
| V^{PC} | Volume inside the peritoneal cavity | L |

In a membrane system permeable only to water (for example Aquaporins), the reflection coefficient is unity and therefore

$$Δ {π}_{O s m} = RT \cdot ( {C}_{O s m}^{P C} - {C}_{O s m}^{P l s} ) \qquad Equation 10$$

| Term | Description | Units |
|---|---|---|
| Δπ_{Osm} | Crystalloid osmotic pressure | mmHg |

By contrast, a system permeable to both solute and water must be scaled by the reflection coefficient. The effective osmotic pressure developed across the membrane, driving the volume flow, may be expressed by combining Equations 4 and 5, yielding:

$$Δ {π}_{O s m}^{Eff} = {σ}^{\tilde{\_}} \cdot {Δπ}_{O s m} \qquad Equation 11$$

where
Δπ_{Osm} is the unscaled differential osmotic pressure across the peritoneal membrane due to the osmoles which could be scaled by aggregated reflection coefficient \overline{\tilde{σ}} to the effective crystalloid osmotic pressure through the peritoneal membrane only considering osmolarities.

### 3.4.3 Osmolarity of Plasma

In order to determine the osmolarity in the plasma,

$${C}_{Osm}^{P l s}$$

of a blood sample is required. The plasma may then be analyzed using routine hospital laboratory equipment such as an ‘Osmomat’ via the freezing point degradation method for example. This procedure only needs to be done occasionally as plasma osmolality may be assumed constant in an individual patient, particularly in continuous therapies such as PD for a longer period of time. In a healthy subject,

$${C}_{Osm}^{P l s}$$

is around 275 mOsm/L while in CKD patients the range is around 270 mOsm/L up to 320 mOsm/L.

### 3.4.4 Osmolality of Fresh Dialysis Fluid

The osmolality

$${C}_{Osm}^{P D F}$$

of fresh dialysis fluid is known from manufacturing.

| Parameter | Description | Units |
|---|---|---|
| ${C}_{Osm}^{PDF}$ | Osmolarity of the fresh Peritoneal Dialysis Fluid for infusion | mosmol/L |

Typical values are:

| PDF | cₒₛₘ_{_{—}}_{PDF} | Units |
|---|---|---|
| 0.55% Glucose | 296-298 | mOsm/L |
| 1.36% Glucose | 344-345 | mOsm/L |
| 1.5% Glucose | 344-347 | mOsm/L |
| 2.27% Glucose | 395-396 | mOsm/L |
| 2.5% Glucose | 394-398 | mOsm/L |
| 3.86% Glucose | 483-484 | mOsm/L |
| 4.25% Glucose | 482-486 | mOsm/L |
| Amino Acid | 365 | mOsm/L |
| 7.5% Polyglucose | 284-301 (additional colloids | mOsm/L |
| | included in the PDF) | (mmol/L) |

### 4 Ultrafiltration Model for Low Molecular Weight Osmotic Agents Based Peritoneal Dialysis Fluids Such as Glucose, Amino Acids Etc. (sUFM1)

The first simplified ultrafiltration model (sUFM1) describes the dynamics of peritoneal cavity volume V^{PC} and the number of osmoles

$${N}_{Osm}^{P C}$$

in the cavity, based on basic principles of volume and mass balance, taking into account transport processes like diffusion, convection and absorption.

### 4.1.1 Parameterisation of the PD Cycle

The dynamics of our model is defined independently for the different phases of the PD cycle as illustrated in FIG. 7.

To obtain an efficient description, we define the start and end time points of the phases of a PD cycle and the volume changes during these phases as follows:

| PD | | | |
|---|---|---|---|
| cycle | | | Volume |
| phase | Start time | End time | change |
| Fill | t₀ | t₁ | V_{Fill} |
| phase | Start of the | | Fill volume |
| | cycle (fill | | [L] |
| | procedure), | | |
| | time point | | |
| | before | | |
| | instillation | | |
| | of PD fluid. | | |
| Dwell | t₁ | t₂ | V_{UF} |
| phase | Start of the | | Ultrafiltration |
| | dwell, time | | volume [L] |
| | point after | | |
| | instillation | | |
| | of PD fluid. | | |
| Drain | t₂ | t₃ | V_{Drain} |
| phase | Start of the | End of | Drain |
| | drain | the PD | volume [L] |
| | phase | cycle | |
| | before | after | |
| | drainage of | drainage | |
| | PD fluid | of PD | |
| | and end of | fluid. | |
| | the dwell of | | |
| | a PD cycle. | | |
| | | | ${V}_{Res}^{PC}$ |
| | | | Residual |
| | | | cavity |
| | | | volume |

The end of the drain phase at t=t₃ marks the beginning of the fill phase of the next cycle.

### 4.1.2 Temporal Variation of the Peritoneal Cavity Volume

In our model, the rate of change in cavity volume is given by the balance of several volume fluxes. Within one PD cycle, the volume rate of change is defined piece-wise for different phases of the cycle:

$$\frac{d {V}^{P C}}{d t} = {J}_{M e m} - + \{ \begin{matrix}{J}_{Fill} & {t}_{0} \leq t \leq {t}_{1} \\ 0 & {t}_{1} \leq t \leq {t}_{2} \\ - {J}_{Drain} & {t}_{2} \leq t \leq {t}_{3}\end{matrix} \qquad Equation 12$$

Eq. 12 applies to a complete PD cycle, i.e., over the range t₀≤t≤t₃ as defined in FIG. 7. The initial conditions for the cycle are defined at t₀.

| Term | Meaning |
|---|---|
| $\frac{{dV}^{PC}}{dt}$ | Rate of change in cavity volume with time during the cycle [L/min] |
| J_{Tot} | Total net flow rate [L/min] |
| J_{Mem} | Flow rate through the peritoneal membrane [L/min] |
| [symbol] | Combined volume flow rate due to the differential |
| | colloid osmotic pressure across the wall of the |
| | peritoneal membrane and lymphatic drainage of |
| | the peritoneal cavity. Specific for an individual |
| | patient and assumed to be constant in the short |
| | term (weeks). |
| J_{Fill} | Fresh dialysate inflow rate during the fill phase |
| | [L/min] |
| J_{Drain} | Cavity volume outflow rate during the drain phase |
| | [L/min] |

The flow through the peritoneal membrane denoted as J_{Mem} is the dominating contribution to the total volume flux which is driven by hydrostatic and crystalloid osmotic pressure during the dwell leading to UFV:

$${J}_{M e m} = - \cdot [ - {σ}^{\tilde{\_}} \cdot RT \cdot ( \frac{{N}_{O s m}^{P C}}{{V}^{P C}} - {C}_{O s m}^{P l s} ) ] \qquad Equation 13$$

| Parameter | Description | Units |
|---|---|---|
| [symbol] | Effective hydraulic conductance. Regarded | L/min/mmHg |
| | as the overall flow through the peritoneal | |
| | membrane per unit pressure across the wall | |
| | of the peritoneal membrane. Specific for an | |
| | individual patient and dependent on the | |
| | number of active pores in the peritoneal | |
| | membrane | |

This equation represents a modification of the formulation described by [Bengt Rippe, Gunnar Stelin, Börje Haraldsson, Computer simulations of peritoneal fluid transport in CAPD, Kidney International, Volume 40, Issue 2, 1991, Pages 315-325, ISSN 0085-2538, https://doi.org/10.1038/ki.1991.216. (https://www.sciencedirect.com/science/article/pii/S0085253815573067)]. The patient specific parameters in Eq 13 are the effective hydraulic conductance and the aggregated reflection coefficient.

The flow [symbol] is a patient-specific parameter, given by the sum of the lymphatic absorption rate and the volume flux due to colloid osmotic pressure caused by proteins in the blood plasma.

The flows J_{Fill} and J_{Drain} are defined over the ranges t₀≤t≤t₁ and t₂≤t≤t₃, respectively, and are zero-valued elsewhere. Within different phases, the flow rates are constant and given by the rates with which the pump of the cycler fills and drains the patient's cavity during standard treatment, respectively. Total fill and drain volumes depend on the time the pump runs.

The fill and drain volumes and the cavity volume V^{PC} at the start and end of the respective phases of the PD cycle result from the above ODEs:

$${V}_{Fill} = {J}_{F i l l} \cdot ( {t}_{1} - {t}_{0} ) \qquad Equation 14$$

$${V}_{D r a i n} = {J}_{D r a i n} \cdot ( {t}_{3} - {t}_{2} )$$

$${V}^{P C} ( {t}_{0} ) = {V}_{Res}^{P C}$$

$${V}^{P C} ( {t}_{1} ) = {V}^{P C} ( {t}_{0} ) + {V}_{Fill} + ( {J}_{M e m} - ) \cdot ( {t}_{1} - {t}_{0} )$$

$${V}_{U F} = ( {J}_{M e m} - ) \cdot ( {t}_{2} - {t}_{1} )$$

$${V}^{P C} ( {t}_{2} ) = {V}^{P C} ( {t}_{1} ) + {V}_{U F}$$

$${V}^{P C} ( {t}_{3} ) = {V}^{P C} ( {t}_{2} ) - {V}_{D r a i n} + ( {J}_{M e m} - ) \cdot ( {t}_{3} - {t}_{2} )$$

### 4.1.3 Temporal Variation of the Osmols in the Peritoneal Cavity

The rate of change of the number of osmoles in the peritoneal cavity during the cycle includes contributions from diffusion, convection (via ultrafiltration) and absorption (lymph and blood proteins) as well as the fill and drain behaviour, which changes the amount of osmotically active substances,

$$\frac{d {N}_{Osm}^{P C}}{d t} = - \cdot ( \frac{{N}_{Osm}^{P c}}{{V}^{PC}} - {C}_{Osm}^{P l s} ) + {J}_{M e m} \cdot {C}_{Osm}^{P l s} - \cdot \frac{{N}_{Osm}^{PC}}{{V}^{P c}} + {J}_{F i l l} \cdot {C}_{Osm}^{P D F} - {J}_{D r a i n} \cdot \frac{{N}_{Osm}^{PC}}{{V}^{P c}} \qquad Equation 15$$

Here,

$${C}_{Osm}^{Pls} and {C}_{Osm}^{PDF}$$

are the osmolarities of the blood plasma and the osmolarity of fresh dialysis fluid, respectively, as introduced in Sections 3.4.2 and 3.4.4.

| Parameter | Description | Units |
|---|---|---|
| [symbol] | Overall permeance of the peritoneal membrane | L/min |
| | to osmoles due to the presence of solutes | |
| | including crystalloids. May be related to | |
| | the overall mass transfer area coefficient | |
| | (MTAC) for all osmoles and determines the | |
| | rate at which solutes permeate the peritoneal | |
| | membrane by diffusive processes. | |

In this ODE the overall permeance of the peritoneal membrane to osmoles is introduced as patient specific membrane parameter.

### 4.2 Schematic Representation and Vector Field Sketch of the ODE System

FIG. 8 summarises which initial conditions and patient-specific membrane parameters and relations are needed to simulate the desired time-dependent state variables. FIG. 9 shows the vector field of the ODE system: examples for the model gradients at t_1 as a function of osmolarity and peritoneal cavity volume. The dotted vertical line shows the start volume in the peritoneal cavity, the horizontal line shows a blood plasma osmolarity of 275 mOsm/L for reference. Solid curves show trajectories of the system over a time of 240 min for a 1.5% PDF (and 4.25% PDF ( ), respectively.

### 4.3 Initial Conditions

How the initial conditions for the model are set generally depends on the scenario to be simulation and may differ for parameter identification or prediction.
For the case of continuous ambulatory peritoneal dialysis (CAPD), there is an unknown in- and outflow of the dialysate due to the cavity. In this case, it is advisable to start the model at t=t₁ (Section 4.3.1).
If the flows are known (by means of a cycler with known fill and drain flows, for example), the calculation should start at t=t₀ (Section 4.3.2).

### 4.3.1 Initial Concentration of Osmoles and Volume, Unknown Fill Flow Rate

In conventional CAPD treatments the fill volume (instilled volume), V_{Fill}, can be measured by weighing the bag of PD fluid pre and post instillation. Under gravity, the fill flow rate, J_{Fill}, varies during the fill phase and is not known with any accuracy (unless flow sensing is introduced in the patient line). In the general case where a residual volume

$${V}_{Res}^{P C}$$

is present in the peritoneal cavity, the number of osmoles at the end of the fill phase can be determined as

$${N}_{O s m}^{P C} ( {t}_{1} ) = {C}_{O s m}^{P C} ( {t}_{0} ) \cdot {V}_{Res}^{P C} + {C}_{O S m}^{P D F} \cdot {V}_{Fill} \qquad Equation 16$$

where

$${C}_{Osm}^{P C} and {C}_{O s m}^{P D F}$$

are the concentrations of osmoles in the peritoneal cavity at time t₀ (start of the fill phase) and the concentration of fresh PD fluid respectively.

Under the assumption that the cavity osmolarity is fully equilibrated with the blood plasma (which is measured rather rarely), we may replace

$${C}_{Osm}^{PC} ( {t}_{0} ) with {C}_{Osm}^{Pls}$$

and assume that the plasma osmolarity ends up on a constant level due to metabolization (on the time scale of weeks at least),

$${N}_{O s m}^{P C} ( {t}_{1} ) = {C}_{O s m}^{P l s} \cdot {V}_{Res}^{P C} + {C}_{Osm}^{P D F} \cdot {V}_{Fill} \qquad Equation 17$$

The initial volume in the peritoneal cavity at the end of the fill phase is given by

$${V}^{P C} ( {t}_{1} ) = {V}_{Fill} + {V}_{Res}^{P C} \qquad Equation 18$$

If the peritoneal cavity is completely empty (dry) at the start of the fill phase, then

$${V}_{Res}^{P C} = 0$$

and Eq. 17 and Eq. 18 may be simplified accordingly. In this case, the simulation and the parameter identification start at t=t₁.

### 4.3.2 Initial Concentration of Osmoles and Volume, Known Fill and Drain Flow Rate

The combined duration of the fill and drain phases of a PD cycle can easily account for 30 mins or more during which transport processes persist. Consequently, a more general model of PD transport processes determines the initial conditions at the start of the PD cycle (t=t₀), rather than solely during dwell phase. The number of osmoles at t₀ is given by

$${N}_{O s m}^{P C} ( {t}_{0} ) = {C}_{O s m}^{P C} ( {t}_{0} ) \cdot {V}_{Res}^{P C} \approx {C}_{O s m}^{P l s} \cdot {V}_{Res}^{P C} \qquad Equation 19$$

And the initial volume is

$${V}^{P C} ( {t}_{0} ) = {V}_{Res}^{P C} \qquad Equation 20$$

In a PD treatment system where the fill flow rate J_{Fill} is known, either by a suitable sensor in the patient line or by APD, this flux may be directly included in the governing equations (Eq. 12 & Eq. 15) describing the transport kinetics. This allows the variation in the number of osmoles and cavity volume during fill and drain to be taken into account.

If the peritoneal cavity is completely empty (dry) at the start of the fill phase, then

$${V}_{Res}^{P C} = 0$$

and both Eq. 19 and Eq. 20 equate to zero.

### 4.4 Evaluation of the sUFM1 With Experimental Data From Literature

Fitting the model to experimental data (described in section 7.2) of a patient study [Gunnar Stelin, Bengt Rippe, A phenomenological interpretation of the variation in dialysate volume with dwell time in CAPD, Kidney International, Volume 38, Issue 3, 1990, Pages 465-472, ISSN 0085-2538, https://doi.org/10.1038/ki.1990.227. (https://www.sciencedirect.com/science/article/pii/S0085253815349899) leads to the following patient-specific membrane fit parameters for the sUFM1 (Twardowski P/V characteristics used for this evaluation from section 3.3.1):

| [symbol] | [symbol] | [symbol] | \overline{\tilde{σ}} |
|---|---|---|---|
| ~0.1 ml/min/mmHg | ~13.7 ml/min | ~1.5 ml/min | ~0.0192 |

Comparison to the parameters of Rippe's TPM [Bengt Rippe, Gunnar Stelin, Börje Haraldsson, Computer simulations of peritoneal fluid transport in CAPD, Kidney International, Volume 40, Issue 2, 1991, Pages 315-325, ISSN 0085-2538, https://doi.org/10.1038/ki.1991.216. https://www.sciencedirect.com/science/article/pii/S0085253815573067)] which are somehow comparable in the dimension of there units:

| LₚS | P_{Glucose}S | L | σ_{Glucose} |
|---|---|---|---|
| 0.082 ml/min/mmHg | 15.5 ml/min | 0.3 ml/min | 0.043 |

The hydraulic conductance LₚS in both models is the major common parameter by comparing both models' parameters. The permeability P_{Glucose}S to Glucose which has dominating influence on the ODE-System of the Rippe TPM was here selected for comparison against the permeability of osmols [symbol] same holds for the reflection coefficient. The Lymph absorption L is less than [symbol] like expected because of the additional flow partition of the colloidal pressure gradient induced flow. For conclusion, the fit parameters of the new model seem to be in the right range and have suitable dimension of the parameter's units.

A comparison of the model fit to the UF Volume for the dwell phase is shown in FIG. 10.

Once the model is fit to patient-specific data, the model can be used to predict the next cycles for a variety of initial conditions.

### 5 Ultrafiltration Model Including Dialysis Fluids With Osmotic Agents of Larger Molecular Weight and Mixtures of Different Molecular Weights (sUFM2)

Our second simplified ultrafiltration model (sUFM2) builds on the same principles as sUFM1 but in addition the transport of colloids and their osmotic pressure were taken into account and described by a third dynamic equation.

### 5.1.1 Colloid Osmotic Pressure Relationship of Polymers and Macromolecules (Such as Polyglucose, Albumin Etc.)

We now describe a method to effectively compute the colloid osmotic pressure produced by larger molecules in the peritoneal cavity. To this end, we compute their concentration and describe an effective way to capture its functional relationship with the corresponding colloid osmotic pressure.

Polymers with different degrees of polymerisation (such as Icodextrin and Albumin) contain polymer chains of different lengths, which affects their contribution to the colloid osmotic pressure.

Next to the mean molar mass (or “number average molar mass”), defined by

$$\overline{M} = \frac{{\sum }_{i} {N}_{i} {M}_{i}}{{\sum }_{i} {N}_{i}} \qquad Equation 21$$

where Nᵢ is the number of molecules of molecular mass Mᵢ, the “mass average molar mass” M̅_̅{̅w̅}̅ (also termed “weight average molar mass”) is a complementary way of characterising the average of the molar mass distribution of polymer chains with different degrees of polymerisation (FIG. 11),

$$\overline{{M}_{w}} = \frac{{\sum }_{i} {N}_{i} {M}_{i}^{2}}{{\sum }_{i} {N}_{i} {M}_{i}} \qquad Equation 22$$

The mass average molar mass puts emphasis on larger molecules. It can be determined by static light scattering, small angle neutron scattering, X-ray scattering, and sedimentation velocity [https://en.wikipedia.org/wiki/Molar_mass_distribution].

Polyglucose solutions (for example Icodextrin) have a weight average molar mass [symbol] between 13 000 and 19 000 Daltons (measured including all crystalloids and colloids).

FIG. 11 is a schematical representation of the molecular weight distribution of a Polydextrin solution

Given the fill volume V_{Fill}, its density ρ_{V}_{_{Fill}} and the fraction ω_{Col} of colloids within the fill solution, the total colloidal mass is given by

$${m}_{c o l} = {V}_{F i l l} \cdot {ρ}_{{V}_{F i l l}} \cdot {ω}_{C o l} \qquad Equation 23$$

and the weight average amount of substance is given by

$$\overline{{N}_{C o l}^{P C}} = \frac{{m}_{c o l}}{\overline{{M}_{ω}}} \qquad Equation 24$$

from which the colloidal concentration follows as

$${C}_{C o l}^{P C} = \frac{\overline{{N}_{C o l}^{P C}}}{{V}^{PC}} \qquad Equation 25$$

| Term | Description | Units |
|---|---|---|
| m_{col} | Mass of | g |
| | colloid | |
| M̅_̅{̅ω̅}̅ | Weight | g/mmol |
| | average | |
| | molar mass | |
| ${C}_{Col}^{PC}$ | Concentration of colloids in | mmol/L |
| | the peritoneal | |
| | cavity | |
| $\overline{{N}_{Col}^{PC}}$ | Weight average | mmol |
| | amount of | |
| | osmotically | |
| | active | |
| | colloidal | |
| | molecules in | |
| | the peritoneal | |
| | cavity | |
| ρ_{V}_{_{Fill}} | Density of the | kg/L |
| | fill volume | |
| ω_{Col} | Mass | % |
| | percentage of | |
| | the colloid | |
| | regarding the | |
| | fill solution | |

Given this method to calculate the colloid concentration in the peritoneal cavity, we turn to the relationship between pressure and concentration. The functional relationship can be effectively captured by a phenomenological approach, in which a polynomial (for example linear or quadratic [From Scatchard, et al. summarized by Landis, E. M. and Pappenheimer, J. R. (1963) In Handbook of Physiology 2, Circulation III, (eds W. F. Hamilton and P. Dow, American Physiological Society, Washington, pp. 961-1034]) is fit to a set of calibration measurements.

Phenomenological relationship of the colloid osmotic pressure and the concentration with a calibration curve created of measurements via for example an Oncometer (FIG. 12).

Here we use a quadratic polynomial to express the colloid osmotic pressure

$${\prod }_{Col}^{P C}$$

as a function of the colloid concentration

$${C}_{Col}^{P C} ,$$

$${\prod }_{C o l}^{P C} ( {C}_{C o l}^{P C} ) = {a}_{1} + {a}_{2} * {C}_{C o l}^{P C} + {a}_{3} * {C}_{C o l}^{P {C}^{2}} \qquad Equation 26$$

| Parameter | Description | Units |
|---|---|---|
| ${\prod }_{Col}^{PC}$ | Colloid osmotic pressure produced by the larger molecules in the peritoneal | mmHg |
| | cavity | |
| a₁, a₂, a₃ | Parameters representing the | |
| | relationship of colloid osmotic | |
| | pressure and colloidal concentration | |

The parameters aᵢ can be determined by fits to measured calibration curves as shown in FIG. 12 and Eq. 26 for the different polymers (Albumin, Icodextrin etc.).

The method mentioned in this section could be used for example for glucose polymers like Icodextrin ( [symbol] ≈16,624 g/mmol) and Albumin ( [symbol] ≈60 g/mmol).

### 5.1 Contribution of the Plasma Proteins

This section describes a method to calculate colloid osmotic pressure produced by the proteins in the patient's interstitium by assuming a functional relationship to the blood plasma osmotic pressure

The largest part of the osmoles is coming from the matrix solution as the basis of the PDF (entire dialysate solution without any osmotic agent (OA) introduced) by osmolarity of matrix of 275 mOsm/L. The entire osmolarity of the Icodextrin solution is around 300 mOsm/L (as measured or as reported on the bag) as initial concentration of colloid in the PDF, on the assumption that the molecular weight average represents MAINLY colloids!

We assume the colloid osmotic pressure of the blood plasma to be largely constant,

$${\prod }_{C o l}^{P L s} \approx const . \qquad Equation 27$$

| Parameter | Description | Units |
|---|---|---|
| ${\prod }_{Col}^{Pls}$ | Colloid osmotic pressure produced by the proteins in the patient's | mmHg |
| | blood plasma | |

If oncotic pressure is related to the interstitial protein concentration, then [Plasma protein osmotic pressure equations for humans, Johan Ahlqvist, Journal of Applied Physiology 2003 94:3, 1288-1289; Joseph Feher, 5.10—The Microcirculation and Solute Exchange, Editor(s): Joseph Feher, Quantitative Human Physiology (Second Edition), Academic Press, 2012, Pages 578-588, ISBN 9780128008836,. (https://www.sciencedirect.com/science/article/pii/B9780128008836000550)].

To determine the interstitial colloid osmotic pressure we use somehow

$${\prod }_{Col}^{Int} = f ( {\prod }_{Col}^{P l s} ) ,$$

but in a first evaluation

$${\prod }_{Col}^{Int} = \frac{2}{3} \cdot {\prod }_{Col}^{P l s}$$

was used for further calculations.

### 5.1.1 Or Could be Measured With a Blood Sample With Measuring the Osmolality and Oncotic Pressure of the Blood Plasma. Temporal Variation of the Peritoneal Cavity Volume

The rate of change in peritoneal cavity volume follows the same basic principles as the one used in sUFM1. However, the effects of lymph absorption and colloidal effects are treated separately:

$$\frac{d {V}^{P C}}{d t} = {J}_{Mem} − + \{ \begin{matrix}{J}_{Fill} & {t}_{0} \leq t \leq {t}_{1} \\ 0 & {t}_{1} \leq t \leq {t}_{2} \\ − {J}_{Drain} & {t}_{2} \leq t \leq {t}_{3}\end{matrix} \qquad Equation 28$$

| Parameter | Description | Units |
|---|---|---|
| [symbol] | Patient-specific lymphatic absorption | L |
| | All other terms analogous to sUFM1. | |

For sUFM2, the membrane flux J_{Mem} now comprises colloidal effects,

$${J}_{Mem} = - \cdot [ - {σ}^{\tilde{=}} \cdot RT \cdot ( \frac{{N}_{Osm}^{PC}}{{V}^{PC}} - {C}_{Osm}^{Pls} ) - ( {\prod }_{Col}^{PC} - {\prod }_{Col}^{Int} ) ] \qquad Equation 29$$

The value of the term

$${Π}_{Col}^{Int}$$

is patient specific and assumed to be around two third of the colloidal osmotic pressure in the blood plasma. This could be measured or assumed for a population of patients as constant and used from publications. Rippe in his model used a colloid osmotic pressure of the blood plasma of 22 mmHg for example. In a first try the same blood colloid osmotic pressure was assumed and scaled as mentioned for the interstitium.

| Parameter | Description | Units |
|---|---|---|
| ${\prod }_{Col}^{PC}$ | Colloid osmotic | mmHg |
| | pressure | |
| | produced | |
| | by the | |
| | proteins in | |
| | the | |
| | patient's | |
| | peritoneal | |
| | cavity | |
| | (Section | |
| | 5.1.1) | |
| ${\prod }_{Col}^{Int}$ | Colloid osmotic | mmHg |
| | pressure | |
| | produced | |
| | by the | |
| | proteins in | |
| | the | |
| | patient's | |
| | interstitium | |

Here the colloid osmotic pressure from the blood depending on proteins in the plasma is taken into account as a large influencing factor.

### 5.1.2 Temporal Variation of the Crystalloid Osmols in the Peritoneal Cavity

The rate of change of the number of osmoles in the peritoneal cavity is modified accordingly,

$$\frac{d {N}_{Osm}^{P C}}{d t} = - \cdot ( \frac{{N}_{Osm}^{PC}}{{V}^{PC}} - {C}_{Osm}^{P l s} ) + {J}_{M e m} \cdot {C}_{Osm}^{P l s} - \cdot \frac{{N}_{Osm}^{PC}}{{V}^{PC}} + {J}_{F i l l} \cdot {C}_{Osm}^{P D F} - {J}_{D r a i n} \cdot \frac{{N}_{Osm}^{PC}}{{V}^{PC}} \qquad Equation 30$$

### 5.1.3 Temporal Variation of the Weight Average Colloid Mols in the Peritoneal Cavity

For sUFM2, we introduce an additional mass transport equation describing the rate of change of the amount of osmotically active colloidal molecules in the peritoneal cavity,

$$\frac{\overline{{dN}_{C o l}^{P C}}}{d t} = - \cdot \frac{\overline{{N}_{C o l}^{P C}}}{{V}^{P C}} + {J}_{Fill} \cdot {C}_{C o l}^{P D F} - {J}_{D r a i n} \cdot \frac{\overline{{N}_{C o l}^{P C}}}{{V}^{P C}} \qquad Equation 31$$

| Parameter | Description | Units |
|---|---|---|
| ${C}_{Col}^{PDF}$ | Concentration of the colloids in the fresh PDF | mmol/L |

Here the assumption is made that during the dwell phase, colloid substance in the cavity is only decreasing by lymph absorption. Because of their size, colloids cannot pass through the small pores which are the majority of the pores and the leakage through large pores is assumed to be negligible.

### 5.1.4 Schematic Representation and Vector Field Sketch of the ODE System

FIG. 13 summarises which initial conditions and patient-specific membrane parameters and relations are needed to simulate the desired time-dependent state variables.

The vector field sketch shown in FIG. 14 demonstrates how the gradients at t₁ look like as a function of osmolarity, colloidal mass and peritoneal cavity volume. The solid and curves show how the system changes over time (8 h) for a 7% Polydextrin PDF and 7.5% Polydextrin PDF, respectively.

### 5.1.5 Initial Concentration of Osmoles, Colloid Concentration and Volume, Unknown Fill Flow Rate

In conventional CAPD treatments the fill volume (instilled volume), V_{Fill}, can be measured by weighing the bag of PDF pre and post instillation. Under gravity, the fill flow rate, J_{Fill}, varies during the fill phase and is not known. In the general case where a residual volume

$${V}_{Res}^{PC}$$

is present in the peritoneal cavity, the number of osmoles and fill volume at the end of the fill phase can be calculated in the same way as for sUFM1 to obtain the initial conditions given at t=t₁.

The additional ODE Eq. X introduced to describe the amount of colloids requires a further initial condition for the weight average amount of osmotically active colloidal molecules inside the peritoneal cavity:

$$\overline{{N}_{C o l}^{P C}} ( {t}_{1} ) = {C}_{C o l}^{P C} ( {t}_{0} ) \cdot {V}_{Res}^{P C} + {C}_{C o l}^{P D F} \cdot {V}_{Fill} \qquad Equation 32$$

where

$${C}_{C o l}^{P C} and {C}_{Col}^{P D F}$$

are the concentrations of colloids in the peritoneal cavity at time t₀ (start of the fill phase) and the concentration of fresh PD fluid, respectively.

If the peritoneal cavity is completely empty (dry) at the start of the fill phase, then

$${V}_{Res}^{P C} = 0$$

and Eq. 32 and may be simplified accordingly.

In this case simulation and parameter identification starts at t=t₁.

### 5.1.6 Initial Concentration of Osmoles, Colloid Concentration and Volume, Known Fill and Drain Flow Rate

The number of osmoles and fill volume at the end of the fill phase can be calculated in the same way as for sUFM1 and hold for the initial conditions at t=t₁ like above.

The amount of osmotically active colloidal molecules at t₀ is

$$\overline{{N}_{Col}^{PC}} ( {t}_{0} ) = {C}_{Col}^{PC} ( {t}_{0} ) \cdot {V}_{Res}^{PC} \qquad Equation 33$$

In a PD treatment system where the fill flow rate J_{Fill} is known, this variable may be included in the set of governing equations describing the transport kinetics. This allows the variation in the number of osmoles and cavity volume during fill and drain to be taken into account.

If the peritoneal cavity is completely empty (dry) at the start of the fill phase, then

$${V}_{Res}^{PC} = 0$$

and Eq. 33 equate to zero.

Plasma proteins and colloids contained in some PD fluids such as polyglucose lead to fluid transport across the membrane due to oncotic pressure. Colloids contribute negligible osmoles, the majority coming from the crystalloids.

### 5.1 Example Simulation of the sUFM2

FIG. 15 shows a simulation of a dwell with a polyglucose solution as a numerical example for the UF Volume for the dwell time (t₁≤t≤t₂) using membrane parameters used by Rippe et al. in their three-pore model (TPM) [Bengt Rippe, Gunnar Stelin, Börje Haraldsson, Computer simulations of peritoneal fluid transport in CAPD, Kidney International, Volume 40, Issue 2, 1991, Pages 315-325, ISSN 0085-2538, https://doi.org/10.1038/ki.1991.216. https://www.sciencedirect.com/science/article/pii/S0085253815573067)] that we identified with the corresponding parameters in our sUFM2:

| | | | | |
|---|---|---|---|---|
| sUFM2 | [symbol] | [symbol] | [symbol] | \overline{\tilde{σ}} |
| Rippe TPM | LₚS | P_{Glucose}S | L | σ_{Glucose} |
| Value | 0.082 | 15.5 | 0.3 | 0.043 |
| | ml/min/mm Hg | ml/min | ml/min | ml/min |

### 6 Further Simplified Model for Single Cycle Prediction sUFM3

By introduction of some additional approximations, sUFM1 may be further simplified yielding sUFM3. Importantly, sUFM3 applies only over the dwell phase, i.e., in the time range t₁≤t≤t₂. This model deals with crystalloid osmotic agent solutions in the same effective way that sUFM1 does.

In contrast to sUFM1 and 2, sUFM3 does not describe the rate of change of osmotically active molecules in the peritoneal cavity

$$( {N}_{Osm}^{PC} )$$

but provides a direct description of the change in the effective osmotic pressure difference

$$Δ {π}_{Osm}^{Eff}$$

that drives the flow through the peritoneal membrane.

sUFM3 is given by the following pair of ordinary differential equations,

$$\frac{{dV}^{PC}}{dt} = - \cdot [ - Δ {π}_{Osm}^{Eff} ] - \qquad Equation 34$$

$$\frac{d Δ {π}_{Osm}^{Eff}}{dt} = - \cdot {Δπ}_{Osm}^{Eff} \qquad Equation 35$$

| | | |
|---|---|---|
| Parameter | Description | Units |

| | |
|---|---|
| [symbol] | Patient specific dissipation coefficient for |
| | the crystalloid osmotic pressure gradient |

The rate of crystalloid dissipation during the dwell phase is determined by crystalloid transport across the membrane and the dilution of crystalloids by the ultrafiltration volume appearing in the peritoneal cavity. If the crystalloid dissipation is approximated as a first-order process, the decay constant becomes the patient parameter denoted by [symbol] .

### 6.1.1 Initial Osmotic Pressure Gradient and Volume

In the general case where, residual volume is present in the peritoneal cavity,

$${V}_{Res}^{PC} ,$$

the fill volume at the end of the fill phase can be calculated in the same way as for sUFM1 to obtain the initial conditions at t=t₁.

The additional ODE describing the osmotic pressure gradient

$$Δ {π}_{Osm}^{Eff}$$

requires an initial condition for the effective osmotic pressure difference at the start of the dwell at t=t₁:

$$Δ {π}_{Osm}^{Eff} ( {t}_{1} ) = \qquad Equation 36$$

| Term | Description | Units |
|---|---|---|
| | Patient- specific | mmHg |
| | osmotic | |
| | pressure | |
| | gradient at the | |
| | start of the | |
| | dwell as a | |
| | function of the | |
| | PDF OA | |
| | concentration. | |
| | (In this model, | |
| | the initial | |
| | condition | |
| | becomes a | |
| | patient- | |
| | specific | |
| | parameter.) | |

### 6.1.2 Extraction of the Aggregated Reflection Coefficient \overline{\tilde{σ}}

If there is no information available about the characteristic of the patient's membrane at the beginning of the treatment, the aggregate reflection coefficient \overline{\tilde{σ}} is unknown.

However, we may infer the reflection coefficient using information about blood plasma and peritoneal cavity fluid osmolarity

$$( {C}_{Osm}^{Pls} and {C}_{Osm}^{PC} ,$$

respectively) at the start of the dwell. First, we calculate a fictitious osmotic pressure gradient Δπ_{Osm}(t₁) under the assumption that the membrane is only permeable to water (in which case the reflection coefficient is unity),

$$Δ {π}_{Osm} ( {t}_{1} ) = RT \cdot ( {C}_{Osm}^{PC} ( {t}_{1} ) - {C}_{Osm}^{Pls} ) = RT \cdot ( \frac{{C}_{Osm}^{PDF} \cdot {V}_{Fill} + {C}_{Osm}^{PC} ( {t}_{0} ) \cdot {V}_{Res}^{PC}}{{V}_{Fill} + {V}_{Res}^{PC}} - {C}_{Osm}^{Pls} ) = RT \cdot ( \frac{{C}_{Osm}^{PDF} \cdot {V}_{Fill} + {C}_{Osm}^{Pls} \cdot {V}_{Res}^{PC}}{{V}_{Fill} + {V}_{Res}^{P C}} - {C}_{Osm}^{Pls} ) \qquad Equation 37$$

Then, the effective osmotic pressure for the semipermeable peritoneal membrane at the start of the dwell is given by Δπ_{Osm}(t₁) scaled by the aggregate reflection coefficient:

$$Δ {π}_{Osm}^{Eff} ( {t}_{1} ) = {σ}^{≃} \cdot {Δπ}_{Osm} ( {t}_{1} ) \qquad Equation 38$$

The effective osmotic pressure gradient

which depends on the PDF OA concentration, can be obtained by patient-specific model fits.

Using this method to determine

$$Δ {π}_{Osm}^{Eff} ( {t}_{1} )$$

and calculating Δπ_{Osm}(t₁) via Eq. 37 for different glucose concentrations, the aggregate reflection coefficient \overline{\tilde{σ}} can be inferred as the slope via a linear fit of

$$Δ {π}_{Osm}^{Eff} ( {t}_{1} )$$

as a function of Δπ_{Osm}(t₁), see Eq. 38.

FIG. 16 is a schematic representation of the relationship of unscaled to scaled osmotic pressure if the membrane is semipermeable

$$Δ {π}_{Osm}^{Eff} ( {t}_{1} ) = {σ}^{≃} \cdot Δ {π}_{Osm} ( {t}_{1} ) + Offset \qquad Equation 39$$

### 6.1 Schematic Representation and Vector Field Sketch of the ODE System

The FIG. 17 summarises which initial conditions and patient-specific membrane parameters and relations are needed to simulate the desired time dependent state variables.

The vector field sketch shown in FIG. 18 demonstrates how the gradients at t₁ look like as a function of the effective osmotic pressure gradient trough the peritoneal membrane and peritoneal cavity volume. The solid curves shows how the system changes over time (240 min) for a 1.5% and 4.25% glucose PDF, respectively.

### 6.1 Evaluation of the sUFM3 With Experimental Data From Literature

Fitting the model to experimental data of a patient study [Gunnar Stelin, Bengt Rippe, A phenomenological interpretation of the variation in dialysate volume with dwell time in CAPD, Kidney International, Volume 38, Issue 3, 1990, Pages 465-472, ISSN 0085-2538, https://doi.org/10.1038/ki.1990.227. (https://www.sciencedirect.com/science/article/pii/S0085253815349899) leads to the following patient-specific membrane parameters for the sUFM3 (Twardowski P/V characteristics used for this evaluation):

| ${C}_{Osm}^{PDF}$ | [symbol] | [symbol] | [symbol] | | | [symbol] |
|---|---|---|---|---|---|---|
| 1.36% | ~0.031 | ~5.5 | ~1.6 ml/min | ~124.6 | mmHg | ~0.0478 |
| 3.86% | ml/min/ | | | ~253.63 | mmHg | (Offset |

| | |
|---|---|
| mmHg | ~55.48) |

We compare these parameters to those of Rippe's TPM [Bengt Rippe, Gunnar Stelin, Börje Haraldsson, Computer simulations of peritoneal fluid transport in CAPD, Kidney International, Volume 40, Issue 2, 1991, Pages 315-325, ISSN 0085-2538, https://doi.org/10.1038/ki.1991.216. https://www.sciencedirect.com/science/article/pii/S0085253815573067)]:

| LₚS | P_{Glucose}S | L | σ_{Glucose} |
|---|---|---|---|
| 0.082 ml/min/mmHg | 15.5 ml/min | 0.3 ml/min | 0.043 ml/min |

Best-fit UF Volume for the dwell phase (t₁≤t≤t₂):

FIG. 19 shows patient data to validate sUFM3

### 7 Identification of Patient Specific Peritoneal Membrane Parameters

### 7.1 Parameter Identification Procedure

In the context of first principle models (sUFM's), ‘patient-specific parameters’ characterize properties of an individual patient's membrane and lymphatic system. As highlighted earlier, one necessary use of the UFM's described is to perform the process of parameter identification. The accuracy of future predictions of ultrafiltration volume depend on the stability (constancy) of the patient-specific parameters. Indeed, the constancy of these parameters is valid only for a limited period of time. It is the process of continual update of patient specific parameters (parameter identification) which is central to the capability to track the changes in the behavior of the peritoneal membrane.

The parameter set \tilde{\vec{P}} to be determined through parameter fits depends on the sUFM as follows,

$$= , = = , =$$

| | Description |
|---|---|
| [symbol] [symbol] [symbol] | Patient- |
| | specific |
| | parameter |
| | set |
| | according |
| | to the |
| | relevant |
| | model |
| | SUFM1, |
| | sUFM2 and |
| | SUFM3, |
| | respectively |
| [symbol] | Patient- specific set |
| | of sUFM3's |
| | initial |
| | conditions |
| | for different |
| | PDFs |
| | Effective crystalloid |
| | osmotic |
| | pressure |
| | gradient |
| | due to the |
| | peritoneal |
| | membrane |
| | dependent |
| | on the |
| | PDF's |
| | osmolarity |

Given a specific model type (sUFM1, sUFM2 or sUFM3), the respective patient-specific parameters are identified by fitting the peritoneal cavity volume V^{PC} over at least 2 PD cycles with different initial conditions. To describe the corresponding cost function used for the parameter fit, we indicate the most recent PD cycle by the index k. Accordingly, the j-th cycle before the current cycle is indicated by the index k-j, see FIGS. 20 and 21

FIG. 20 shows consecutive cycles directly followed by each other, and FIG. 21 shows consecutive cycles with a dry phase in between, which allows the residual volume to be reabsorbed again

The cost function for the fit is given by

$${Γ ( \vec{\tilde{P}} ) = {\sum }_{j = 0}^{n} {\sum }_{t = {t}_{0}}^{{t}_{3}} ( t ) - ( t ) ]}^{2}$$

and the best-fit parameter set is defined as the one minimising the cost function,

$${\vec{\tilde{P}}}_{fit} = {arg min}_{{P}^{\vec{˜}}} Γ ( \vec{\tilde{P}} ) .$$

| Term | Description |
|---|---|
| k | Index of the most recent PD cycle |
| n | Total number of PD cycles in the past considered for the parameter |
| | fit |
| [symbol] (t) | Volume inside the intraperitoneal cavity measured directly, |
| | by dilution methods or due to the intraperitoneal pressure |
| | measurement combined with the pressure volume characteristics |
| [symbol] (t) | Volume inside the intraperitoneal cavity simulated by the sUFM's |
| Γ | Cost function used for the parameter fit |

Depending on how continuously the peritoneal dialysis treatment performed, a large number of PD cycles may be used for parameter optimization. In the best case, some of the cycles would have different initial conditions, which leads to a more reliable determination of the parameters. In all cases, a measurement of the blood plasma osmolality

$${C}_{Osm}^{Pls}$$

would be preferred as input for the models.

Summary of the parameter identification procedure:
The model is fitted to a number of PD cycles with different initial conditions, using a method to minimize the deviation between measured and simulated state variables by adjustment of the patient-specific parameters, for example with the method of least squares comparing measured volume (via P-V characteristic) and modelled cavity volume→\tilde{\vec{P}}.
Could also use pressure if the equations are adjusted. But then the measured pressure signal

$${P}_{Meas}^{PC} ( t )$$

need to be fitted to the intraperitoneal hydrostatic pressure of the cavity

for the ODE system.
As a more specific example, the residual cavity volume

$${V}_{Res}^{PC}$$

is subject to variability between cycles due to a number of practical constraints during drainage and any time that elapses between cycles. In case of FIG. 20,

$${V}_{Res}^{PC}$$

is smaller on the previous cycle [k−1] compared with current cycle [k]. This is expressed formally as

$${V}_{Res}^{PC} [ k - 1 ] ( {t}_{0} ) < {V}_{Res}^{PC} [ k ] ( {t}_{0} ) .$$

Using such notation any sequence of cycles may be described including ‘standard’ cycles whereby the peritoneal cavity is fully drained (as far as possible) and ‘tidal’ cycles whereby the peritoneal cavity is partially drained before fresh PDF is instilled.
The same use of variables applies, regardless if the cycles are consecutive or there is a dry phase (empty cavity) between two PD cycles as shown in FIG. 21.

### 7.2 Design of Calibration PD Cycles for sUFM1

In general, the estimation of patient-specific membrane parameters is more robust if an adequately large number of cycles is used and if initial conditions vary between cycles. To achieve this systematically, we describe how sequences of PD cycles dedicated to model calibration can be designed. Such calibration cycles probe the membrane characteristics of a specific patient through different initial conditions and external inputs. They contain an increased amount of information as compared to sequences of identical cycles and thus, lend themselves to parameter estimation.

### 7.2.1 Varying the Amount of Osmotic-Agent (OA) Solution Between Cycles

FIG. 22 shows consecutive cycles using dialysis solutions comprising different amounts of osmotic agent.
1^{st} cycle should start with empty cavity or a known residual volume
1^{st} cycle drain should occur after peak when V^{PC}[k−1](t₂)≈V^{PC}[k−1](t₁)
1^{st} cycle with low glucose to minimise time to 0 UFV
After drainage the weight difference is the residual volume
2^{nd} cycle with high glucose
Drain once peak has been achieved and glucose dissipation is close to zero

### 7.2.2 Varying the Fill Volume Between Cycles

FIG. 23 shows consecutive cycles using different fill volumes.

Two fill volumes in consecutive cycles with V_{Fill}[k−1](t₁)<V_{Fill}[k](t₁) are required whereby both volumes differ by at least several hundred mL. Preferably, these volumes should be the minimum and maximum fill volume of a typical treatment for the respective patient.
1^{st} must start with empty cavity or a known residual volume
1^{st} cycle fills to an arbitrary fill volume V_{Fill}[k−1](t₁)
1^{st} cycle drain should occur after peak when V^{PC}[k−1](t₂)=V_{Fill}[k−1](t₁)
1^{st} cycle with low fill volume V_{Fill}[k−1](t₁) to minimise time to 0 UF volume
After draining the weight difference is the residual volume
2^{nd} cycle with higher fill volume, V_{Fill}[k](t₁)
Drain once peak has been achieved and glucose dissipation is close to zero

### 7.2.3 Varying the Dwell Time Between Cycles

FIG. 24 shows several cycles with different dwell durations while filling the cavity to the same volume and using the same PDF (taking residual volume into account).
Dwell time should differ by at least 30 to 60 min, were the 1^{st} cycle should be the reference. The 2^{nd} cycle should be at least 30 min shorter and the 3^{rd} cycle should be at least 30 min longer.
1^{st} cycle starts with empty cavity or know residual volume
1^{st} cycle drain should occur after peak when V^{PC}[k−2](t₂)=V_{Fill}[k−2](t₁)
After each drain we get the residual volume
2^{nd} cycle drain should occur after peak when V^{PC}[k−1](t₂)>V_{Fill}[k−1](t₁)
3^{rd} cycle drain should occur after peak when V^{PC}[k](t₂)<V_{Fill}[k](t₁)

### 7.2.4 Hybrids (Combinations of Methods)

All of the above methods can be combined in different ways to cater for practical restrictions regarding treatment regimens and improve accuracy of parameter identification. It should be recognised that the above methods are ‘designed experiments’ which in isolation allow a full set of parameters to be obtained in the fewest number of cycles. However, during routine PD treatments there is opportunity for ongoing parameter identification updates, particularly any change which leads to different cycle to cycle variations such as fill volume, glucose composition or dwell time.

### 7.3 Design of Calibration PD Cycles for sUFM2

The general concepts introduced for the design of calibration cycles for sUFM1 apply for sUFM2. FIG. 25 shows an example with a varying fill volume and no consecutive cycles using colloid PDF (ICO=Icodextrin). Between the colloid cycles, cycles using mostly crystalloid PDF (glucose or amino acid) are added to the patient's treatment. For such combinations of different PDFs, the employed model must depend on the used dialysate solution.

### 7.4 Design of Calibration PD Cycles for sUFM3

This simplified model only needs a designed experiment (see FIG. 26) with variation of the OA concentration in the PDF. The initial conditions depend on the fill volume, although the relationship cannot be expressed explicitly. Therefore the influence of the fill volume on the effective crystalloid osmotic pressure (initial conditions) needs to be determined by designed experiments leading to parameter identification.

### 8 Continuous Real-Time Calibration of the Model in Applications

Whenever a predicted cycle is executed, the same scheme for parameter estimation can be used with updated IPP measurements as input. This generates an updated set of fit parameters. Thus, a history of parameters can be generated that are specific to the individual patient. This feedback may be used to further improve the accuracy of the prediction from treatment to treatment by feeding information from previous parameter fits into the current parameter fit, and/or to monitor the robustness of parameter fits (e.g., by comparing by how much parameters change from one cycle to the next.

### 9 Use of the Models for UF Volume Prediction

The models can be used to predict UF volume for any prescription (Fill volume, glucose content and dwell duration)

To use the models to predict UF volume, the following general strategy is implemented:
1. Measurements of patient-specific input data (e.g., previous UF volume and IPP information) for the model are carried out.
2. Patient-specific parameters and relations can then be inferred by fitting the model to the measured data. It is possible to fit parts of the model or the entire model directly to the measured data.
3. The UF Volume is predicted for a variety of prescriptions for the patient using the model, in order to find a prescription that meets UF Volume requirements for the patient. The prediction can also be used on a cycle to cycle basis in order to adapt a prescription to changes in the value of the parameters in the model caused by changes in patient membrane characteristics. The aim is to obtain the best possible treatment.

### 10 Overview of the sUFMs

| | sUFM1 | sUFM2 | sUFM3 |
|---|---|---|---|
| | Multicycle, | Multicycle, | Single dwell, |
| | Low molecular weight OA | Low & high molecular weight OA | Low molecular weight OA |
| Description | Model of consecutive cycles with | Same as 1 but extended for | Alternative to model 1 with |
| | fill, dwell and drain | colloid osmotic solutions such as | the restriction that it applies |
| | Identification of parameters from | polyglucose | only to the dwell phase. |
| | the dwell phases of any two | | Identification with dwell |
| | different cycles | | only. Less accurate in |
| | | | parameter identification. |
| UF Prediction | Dwell only or complete | | |
| | consecutive cycles with different | | |
| | glucose | | |
| Initial Conditions | ${V}^{PC} ( {t}_{0} ) = {V}_{Res}^{PC}$ ${N}_{Osm}^{PC} ( {t}_{0} ) = {V}_{Res}^{PC} \cdot {C}_{Osm}^{Pls}$ | ${V}^{PC} ( {t}_{0} ) = {V}_{Res}^{PC}$ ${N}_{Osm}^{PC} ( {t}_{0} ) = {V}_{Res}^{PC} \cdot {C}_{Osm}^{Pls}$ ${N}_{Col}^{PC} ( {t}_{0} ) = {V}_{Res}^{PC} \cdot {C}_{Col}^{Pls}$ | ${V}^{PC} ( {t}_{1} ) = 0$ $( {t}_{1} ) =$ |
| Reflection | Constant | Constant | Function |
| coefficient | | | |
| Time Range | t₀−t₃ | t₀−t₃ | t₁−t₂ |
| Known information needed (Parameter identification) | $Method 1 ( Residual volume in cavity ) :$ ${V}_{Res}^{PC} ; {C}_{Osm}^{Pls} or$ ${C}_{Osm}^{PC} ; {C}_{Osm}^{PDF} ; {J}_{Fill} ; {J}_{Drain}$ $Method 2 ( empty cavity ) :$ ${C}_{Osm}^{Pls} ; {C}_{Osm}^{PDF} ; {J}_{Fill} ; {J}_{Drain}$ | $Method 1 : {V}_{Res} ; {C}_{Osm}^{PC} or$ ${C}_{Osm}^{Pls} ; {C}_{Col}^{PDF} ; {C}_{Col}^{PDF} ; {J}_{Fill} ; {J}_{Drain}$ $Method 2 : {C}_{Osm}^{Pls} ; {C}_{Col}^{PDF} ; {C}_{Col}^{PDF} ; {J}_{Fill} ; {J}_{Drain}$ | $Method 1 : {V}_{Fill} ; {V}_{Res} ; {C}_{Osm}^{PDF}$ ${C}_{Osm}^{PC} or {C}_{Osm}^{Pls}$ $Method 2 : {V}_{Fill} ; {C}_{Osm}^{PDF} {C}_{Osm}^{Pls}$ |
| Prediction | Multiple cycles with fill, dwell and | Multiple cycles with fill, dwell and | Single cycle dwell if V_{Fill} |
| capability | drain phase | drain phase | and V_{Res} is assumed to be |
| | | | constant |
| Parameter | Use of different initial conditions | Use the standard treatment | Use of 2 or more different |
| identification | cycles (designed exp.) and the | schedule to track the membrane | % Glucose standard PDF |
| | standard treatment schedule to | parameters, mostly this model | (designed exp.) |
| | track the membrane parameters | needs to be combined with | |
| | | sUFM1 | |
| Membrane Parameters | $\vec{{\tilde{P}}_{1}} =$ | $\vec{\tilde{{P}_{2}}} =$ | $\vec{\tilde{{P}_{3}}} =$ $=$ |
| Possible | Glucose, Amino acids, all | Polydextrins, colloid solutions, | Need to adjust in the right |
| PDF's | crystalloid active substances | mixture of Polydextrin with | way for the used PDF with |
| | | crystalloid active substances | some designed cycles, but |
| | | (Glucose, amino acids etc.) | then all crystalloid active |
| | | | substances (Glucose, |
| | | | amino acids etc.) and |
| | | | Polydextrins should be |
| | | | possible after parameter |
| | | | identification |
| Benefits | 2 designed experiments at the | Parameter identification and | 2 designed experiments at |
| | start of treatment needed with | tracking with the applied | the start of treatment |
| | known conditions | treatment schedule possible, | needed |
| | Continuous parameter updates | but at the start of Icodextrin | 2 to 4 initial conditions |
| | and tracking with the applied | treatment, known conditions | needed |
| | treatment schedule possible | are needed | |
| | Accurate | Accurate | |
| | Can predict a series of cycles | Can predict a series of cycles | |
| Drawbacks | 3 to 6 initial conditions needed | 3 to 7 initial conditions needed | Less accurate |
| | | | Prediction of only next |
| | | | cycle |

### 11 Further Modification Possibilities of the Models

sUFM1 and sUFM2 can be extended by further ODEs describing the change of the extracellular volume V^{EV}, the plasma osmolarity

$${N}_{Osm}^{Pls}$$

and the colloid blood concentration

$$\overline{{N}_{col}^{Pls}}$$

to build up a two-pool model:

For both models we use

$${C}_{Osm}^{Pls} = \frac{{N}_{Osm}^{Pls}}{{V}^{EV}}$$

Additional ODEs for sUFM1:

$$\frac{{dV}^{EV}}{dt} = - {J}_{Mem} +$$

$$\frac{{dN}_{Osm}^{Pls}}{dt} = \cdot ( \frac{{N}_{Osm}^{PC}}{{V}^{PC}} - {C}_{Osm}^{Pls} ) - {J}_{Mem} \cdot {C}_{Osm}^{Pls} + \cdot \frac{{N}_{Osm}^{PC}}{{V}^{PC}}$$

The initial conditions for V^{EV} depend on the hydration status of the patient and

$${N}_{Osm}^{Pls}$$

should be measured frequently in blood plasma samples via an Osmomat.

Additional ODEs for the sUFM2:

$$\frac{{dV}^{EV}}{dt} = - {J}_{Mem} +$$

$$\frac{{dN}_{Osm}^{Pls}}{dt} = \cdot ( \frac{{N}_{Osm}^{PC}}{{V}^{PC}} - {C}_{Osm}^{Pls} ) - {J}_{Mem} \cdot {C}_{Osm}^{Pls} + \cdot \frac{{N}_{Osm}^{PC}}{{V}^{PC}}$$

The initial conditions are the same as for the sUFM1, but additionally the amount of plasma colloids, which is dependent on the metabolism of the patient, needs to be extracted from a blood sample via Oncometer measurement.

| Parameter | Description | Units |
|---|---|---|
| ${N}_{Osm}^{Pls}$ | Amount of osmotically active | mosmol/L |
| | molecules in the blood plasma | |
| V^{EV} | Extracellular volume | L |
| $\overline{{N}_{Col}^{Pls}}$ | Weight average amount of colloids | mmol/L |
| | in the blood plasma | |
| ε | Metabolism rate of the patient | L |

### 12 Model Variables

| Term | Meaning | Unit |
|---|---|---|
| [symbol] | Patient-specific relationship between the volume in the | mmHg |
| | peritoneal cavity and the resulting intraperitoneal pressure | |
| ${P}_{Hy}^{PC}$ | Pressure in the intraperitoneal cavity | mmHg |
| ${P}_{0}^{PC}$ | Pressure at the volume V₀ | mmHg |
| V^{PC} | Volume in the intraperitoneal cavity | L |
| ${V}_{0}^{PC}$ | Volume at the pressure P₀ | L |
| | Patient-specific relationship between the volume in the | mmHg |
| | peritoneal cavity and the resulting intraperitoneal pressure | |
| ${P}_{Meas}^{PC} ( t )$ | Quasi-continuous measurement of the intraperitoneal pressure | mmHg L |
| | during fill and drain (dwell) | |
| ${V}_{Meas}^{PC}$ | Quasi-continuous measurement of the intraperitoneal volume | L |
| | during fill and drain | |
| ${P}_{HS}^{Int}$ | Patient-specific interstitial pressure (depends on the hydration | mmHg |
| | status of the patient) | |
| ${C}_{i}^{Pls}$ | Concentration of the i-th solute in the blood plasma | mmol/L |
| ${C}_{i}^{PC}$ | Concentration of the i-th solute in the peritoneal cavity fluid | mmol/L |
| R | Gas constant | kg·m²·S⁻²·mol⁻¹·K-1 |
| T | Temperature | K |
| σ_{i,m} | Reflection coefficient for the i-th solute and the m-th pore size | Dimensionless |
| Δπ^{Eff} | Effective crystalloid osmotic pressure which drives a fluid flow | mmHg |
| | through the peritoneal membrane | |
| [symbol] | Aggregated reflection coefficient (all solutes considered), | Dimensionless |
| | dependent on the active pores in the individual patient | |
| ${C}_{Osm}^{Pls}$ | Osmolarity of the blood plasma | mosmol/L |
| ${C}_{Osm}^{PC}$ | Osmolarity of the peritoneal cavity volume | mosmol/L |
| ${Δπ}_{Osm}^{Eff}$ | Effective crystalloid osmotic pressure which drives a fluid flow | mmHg |
| | through the peritoneal membrane only considering osmoles | |
| ${N}_{Osm}^{PC}$ | Amount of osmotically active molecules in the peritoneal cavity | mosmol |
| V^{PC} | Volume inside the peritoneal cavity | L |
| Δπ_{Osm} | Crystalloid osmotic pressure | mmHg |
| ${C}_{Osm}^{PDF}$ | Osmolarity of the fresh Peritoneal Dialysis Fluid for infusion | mosmol/L |
| $\frac{{dV}^{PC}}{dt}$ | Rate of change in cavity volume with time during the cycle | L/min |
| J_{Tot} | Total net flow rate | L/min |
| J_{Mem} | Flow rate through the peritoneal membrane | L/min |
| [symbol] | Combined volume flow rate due to the differential colloid | L/min |
| | osmotic pressure across the wall of the peritoneal membrane | |
| | and lymphatic drainage of the peritoneal cavity. Specific for an | |
| | individual patient and assumed to be constant in the short term | |
| | (weeks). | |
| J_{Fill} | Fresh dialysate inflow rate during the fill phase | L/min |
| J_{Drain} | Cavity volume outflow rate during the drain phase | L/min |
| t₀ | Start of the cycle (fill procedure), time point before instillation | min |
| | of PD fluid | |
| t₁ | Start of the dwell, time point after instillation of PD fluid | min |
| t₂ | Start of the Drain phase before drainage of PD fluid and end | min |
| | of the dwell of a PD cycle | |
| t₃ | End of the PD cycle after drainage of PD fluid | min |
| V_{Fill} | Fill volume | L |
| V_{Drain} | Drain volume | L |
| V_{UF} | Ultrafiltration volume | L |
| ${V}_{Res}^{PC}$ | Residual cavity volume | L |
| [symbol] | Effective hydraulic conductance. Regarded as the overall flow | L/min/mmHg |
| | through the peritoneal membrane per unit pressure across the | |
| | wall of the peritoneal membrane. Specific for an individual | |
| | patient and dependent on the number of active pores in the | |
| | peritoneal membrane | |
| [symbol] | Overall permeance of the peritoneal membrane to osmoles due | L/min |
| | to the presence of solutes including crystalloids. May be | |
| | related to the overall mass transfer area coefficient (MTAC) | |
| | for all osmoles and determines the rate at which solutes | |
| | permeate the peritoneal membrane by diffusive processes. | |
| m_{col} | Mass of colloid | g |
| M̅_̅{̅w̅}̅ | Weight average molar mass | g/mmol |
| ${C}_{Col}^{PC}$ | Concentration of colloids in the peritoneal cavity | mmol/L |
| $\overline{{N}_{Col}^{PC}}$ | Weight average amount of colloidal active molecules in the peritoneal cavity | mmol |
| ρ_{V}_{_{Fill}} | Density of the fill volume | kg/L |
| ω_{Col} | Mass percentage of the colloid regarding the fill solution | % |
| ${\prod }_{Col}^{PC}$ | Colloid osmotic pressure produced by the larger molecules in the peritoneal cavity | mmHg |
| a₁, a₂, a₃ | Parameters representing the relationship of colloid osmotic | |
| | pressure and colloidal concentration | |
| ${\prod }_{Col}^{Int}$ | Colloid osmotic pressure produced by the proteins in the patient's interstitium | mmHg |
| \tilde{J}_{L} | Patient specific lymphatic absorption | L |
| ${C}_{Col}^{PDF}$ | Concentration of the colloids in the fresh PDF | mmol/L |
| [symbol] | Patient specific dissipation coefficient for the crystalloid | |
| | osmotic pressure gradient | |
| ${\prod }_{Col}^{Pls}$ | Colloid osmotic pressure produced by the proteins in the patient's blood plasma | mmHg |
| [symbol] | Patient-specific osmotic pressure gradient at the start of the | mmHg |
| | dwell (In this model the initial condition becomes a patient- | |
| | specific parameter) | |
| [symbol] [symbol] , [symbol] | Patient-specific parameter set according to the relevant model | |
| | sUFM1, sUFM2 and sUFM3, respectively | |
| | Patient specific set of sUFM3's initial conditions for different PDFs | mmHg |
| | Effective crystalloid osmotic pressure gradient due to the | mmHg |
| | peritoneal membrane, dependent on the PDF's osmolarity | |
| n | Total number of PD cycles in the past considered for the | |
| | parameter fit | |
| [symbol] | Volume inside the intraperitoneal cavity measured directly, by | L |
| | dilution methods or due to the intraperitoneal pressure | |
| | measurement combined with the pressure volume | |
| | characteristics | |
| [symbol] | Volume inside the intraperitoneal cavity simulated by the | L |
| | sUFM's | |
| ${N}_{Osm}^{Pls}$ | Amount of osmotically active molecules in the blood plasma | mosmol/L |
| V^{EV} | Extracellular volume | L |
| $\overline{{N}_{Col}^{PC}}$ | Weight average amount of colloids in the blood plasma | mmol/L |

### 13 Constants

| Term | Value |
|---|---|
| RT | 19.3 |
| ${P}_{HS}^{Int}$ | −3 to 3 |
| ${C}_{Osm}^{Pls}$ | 275 to 325 |
| ε | 0 to 1 |
| ${C}_{Osm}^{PDF}$ | 280 to 500 |
| ${\prod }_{Col}^{PLs}$ | 22 |
| M̅_̅{̅w̅}̅_{_{ICO}} | 16.624 |
| ρ_{V}_{_{Fill}} | ~1 |
| ω_{Ico} | 7.5 |

### 14 Clinical Applications

FIG. 29 depicts clinical applications associated with UFV predictions. UFV predictions 2910 may be determined according to various processes, including statistical (for example, processes described in the 190130 Application) or first principal techniques or models. One or more UFV predictions 2910 may then be used in various clinical applications 2920a-n. Non-limiting examples of clinical applications 2920a-n may include fluid status maintenance 2920a and peritonitis detection (or prediction) 2920b. Accordingly, UFV predictions 2910 may be applied in various techniques of patient treatment.

Regarding the maintenance of hydration status of a patient, the TBW of a subject may undergo diurnal variation, for example, due to ingestion of food and water, urinary output, subject activity, and/or the like. This leads to small random variation in TBW about a nominal value of TBW that the normal kidney function serves to maintain. In kidney failure, more of the fluid that is ingested accumulates in body tissues as excess fluid. Dialysis treatment, or more specifically the process of ultrafiltration, serves to remove this excess fluid.

FIG. 30 depicts graph 3010 of variation in TBW 3020 over a 24-hour period 3025 and removal of excess fluid accumulation by ultrafiltration. Graph 3010 depicts a TBW plot 3040 about a target hydration status 3026. TBW plot 3040 is modified by random fluid gain 3024 and UFV 3021 for each cycle of PD.

An essential basis for any fluid removal strategy is the identification of a hydration target (e.g., 3026 of FIG. 30) for the individual patient. Essentially, the target reflects the hydration that should be achieved if the kidney were to work normally, considering adjustments necessary to mitigate any deleterious effects of comorbidity. The control of ultrafiltration requires both the hydration target and suitable measurements of the patient's hydration status. Multiple measurements of hydration status over several days may be considered. These measurements may be averaged (to compensate for measurement errors) and used to track typical variation in hydration status. The measurement of hydration status may be achieved via a number of possible established methods. Hydration status may be measured at suitable intervals e.g., daily, so that the control loop can compensate for variations in hydration status on a treatment-by-treatment basis. The difference between the hydration target and the current hydration status is the error or the signal that is fed into a controller configured according to some embodiments.

FIG. 31 illustrates an exemplary operating environment in accordance with the present disclosure. More specifically, FIG. 31 depicts a process that uses an inference engine 3101 to determine dose variables 3103. Inference engine 3101 may include a UF prediction module 3140 having a dose variable optimizer 3141. In some embodiments, dose variable optimizer model 3141 may receive a model parameter set 3160 and patient and/or unknown information 3114 as input. Dose variable optimizer model 3141 may operate to adapt dose variables 3103 of a prescription 3102 to minimize an error 3116 between a predicted UFV 3110 and a target UFV 3138. Prescription 3102 may be used for dialysis treatment of patient 3150. Patient hydration statuses may be measured 3124, for instance, daily, to determine a hydration status 3123. A hydration target 3120 may be analyzed against hydration status 3123 using a controller 3121 to determine target UFV (per treatment) 3138. In various embodiments, the controller (e.g., controller 3121) may be, may include, and/or may use a software-based algorithm configured to maintain the hydration status constant or substantially constant, but also to minimize the error (e.g., 3116) between hydration status and hydration target. The output of the controller may be a target UFV (e.g., 3138) for the next PD treatment. The target UFV compensates for changes in hydration status arising from fluid ingestion, urine output (partial kidney function), other insensible losses, and/or the like. The control algorithm may be configured according to various techniques, including, without limitation, a conventional ‘PID’ (proportional, integral, derivative) controller to more sophisticated expert system approaches.

In the case of PD, the UFV target for the next treatment appropriate values of the dose variables for a sequence of PD cycles. The dose variable values are identified by application of rules, that are encapsulated within an ‘inference engine’ (e.g., 3101). The rules decide how the dose variables are manipulated most efficiently. Rules may, for example, seek to adjust only dwell duration in preference to changing glucose composition, while ensuring that other dialysis KPIs are met, such as adequate small solute clearance. Control of hydration is possible, for example, using adequate knowledge pertaining to the behavior of the dwell profile, obtained from historical measurements or suitable assumptions. For instance, control can be achieved without the need necessarily for the more generalizable method of UFV prediction. Where there are insufficient degrees of freedom with dwell duration alone, glucose and/or fill volume may be also be manipulated. In some embodiments, the direction in which glucose or dwell duration are manipulated and their magnitude, may depend on the quality of historical data, underlying assumptions, and/or the like.

If a model of the system is invoked, in this case the UFV prediction model, relating the dose variables to the expected UFV, then significantly improved performance may be achieved with regards to control of the patient's hydration status. For example, because dose variables cannot be controlled in real time; their values must be determined prior to each PD treatment cycle. Thus, inclusion of UFV prediction in the inference engine mitigates the potential for oscillations in hydration status as well as minimizing the error between hydration status and the hydration target. Consequently, the UFV prediction capability has high utility in deciding the best combination of dose variables, especially where information on dwell characteristics under different conditions is poor or assumptions are doubtful. This reduces the reliance on rules and also enables other KPIs to be more easily fulfilled, including, without limitation:
The combination of dose variables that lead to the lowest concentration of glucose, mitigating the effects on long term glucose exposure;
Ensuring sufficient time to achieve adequate small solute clearance; and/or
Avoiding dwell durations that are too short, running the risk of sodium loading that can occur in the early part of the dwell due to sodium sieving properties of the peritoneal membrane.

In some embodiments, there may be no or substantially no restrictions with regarding the administration of the osmotic agent (e.g., glucose). Many PD treatment systems involve the use of bags of differing glucose strength, leading to discreet glucose composition increments. By contrast, a dedicated glucose proportioning system may allow selection of any specific glucose composition within a defined range. The latter approach offers a finer degree of granularity and is useful in situations where there may be restrictions on dwell duration of individual cycles or overall treatment time.

Feedback control according to some embodiments provides better maintenance of hydration status, the potential advantages of which may include, without limitation:
Minimize cardiovascular (CV) risk by maintenance of optimal hydration status;
Elimination of trial and error PD prescriptions;
The enablement of flexible treatments that can adapt to an individual patient's lifestyle, including Quality of Life (QoL) improvement;
Minimizing the need for different bag combinations, simplifying PD treatment;
Minimizing the exposure to the osmotic agent, affording better long-term membrane preservation; and/or
Increase technique survival (as a consequence of reduced CV risk, better QoL and better membrane preservation).

UFV predictions according to some embodiments may be used to determine and/or predict peritonitis and/or signs thereof. Peritonitis (infection of the peritoneal membrane arising from several causes) is a common complication of PD therapy. During an infection, large pores in capillary beds open to increase the release of acute phase proteins such as albumin. This effect may be characterized by a change in the peritoneal membrane transport parameters, particularly in regard to increased dissipation of glucose from the peritoneal cavity, which in turn leads to a reduction in the measured UFV.

The predicted UFV may be obtained from various models described in the present disclosure, the parameters of which are continually updated with data from previous treatments, for instance, via a parameter optimization process (e.g., FIG. 31). It is assumed that high UFV prediction accuracy is achieved by selection of an appropriate regression (statistical) model with sufficient inputs, and interactions between terms as required. Alternatively, UFV may be predicted by any other type of model such as a first principles model. A surrogate marker of peritonitis is obtained by comparing the measured and predicted UFV.

FIG. 32 illustrates an exemplary operating environment in accordance with the present disclosure. More specifically, FIG. 32 depicts a process that includes determining a prescription 3202 with dose variables 3203. In some embodiments, the dose variables 3203 may include a fill volume, fill duration, and/or an osmotic agent concentration. The dose variables 3203 may be included in a design experiment 3212. In various embodiments, design experiment 3212 may include a sequence of PD cycles that involve different combinations of dose variables 3203 performed on a patient 3250 via a PD treatment system 3220. A measured UFV 3236 may be determined for each cycle.

In some embodiments, dose variables 3203 (and unknown factors 3214, such as patient information, time of day, activity levels, etc.) may be provided to a UFV prediction model 3240 configured to generate a predicted UFV 3210. In various embodiments, predicted UFV 3210 may be compared with measured UFV 3236 to determine a probability of peritonitis 3270. In general, in some embodiments, probability of peritonitis 3270 may be a function of or otherwise associated with an error between measured UFV 3236 and predicted UFV 3210.

Under normal circumstances (where no peritonitis is present), the error between the measured and predicted UFV may be small. The higher the difference between the measured and predicted UFV, the greater the probability of peritonitis (e.g., 3270). In some embodiments, the rate of change of measured UFV (improving robustness by minimizing false-positive events) may be monitored. As model parameters are update continually at the end of each PD cycle, various embodiments may include monitoring for sudden change (e.g., a change over a threshold value, percentage, and/or the like) in model parameters.

Where an episode of peritonitis is present, the duration of which may span several days or longer, some embodiments may operate to exclude the treatment data normally used to update model parameters. In other embodiments, where a deviation in UFV or model parameters falls below a ‘peritonitis probability threshold’, then that data may be used to update model parameters.

1. A system for predicting the magnitude of ultrafiltration volume expected in a peritoneal dialysis treatment for an individual patient, the system comprising:
a peritoneal treatment machine configured to perform cycles of a peritoneal treatment performed on the patient according to a prescription by controlling at least one actuator and/or valve of the peritoneal treatment machine, the cycles comprising a fill phase, a dwell phase and a drain phase,
a sensor for repeatedly measuring a sensed value during the treatment performed on the patient,
a controller programmed to predict the magnitude of ultrafiltration volume expected during a treatment performed according to a prescription based on a model of ultrafiltration, and to fit parameter values of the model to the individual patient based on the values measured by the sensor,
wherein the model of ultrafiltration uses a patient-specific aggregated reflection coefficient as a parameter representing the overall average effect of different pores of the peritoneum and of different solutes present in the peritoneal cavity and the blood plasma on the differential crystalloid osmotic pressure between the peritoneal cavity and blood plasma.

2. The system of claim 1, wherein the model uses the aggregated reflection coefficient to describe the overall dependency of the differential crystalloid osmotic pressure between the peritoneal cavity and blood plasma on the difference between the osmolarity of the dialysis solution present in the peritoneal cavity and the osmolarity of the blood plasma.

3. The system of claim 1, wherein the controller is programmed to receive the overall osmolarity of the dialysis solution instilled into the patient as an input to the model of ultrafiltration, the system preferably comprising a user interface configured for inputting a value for the osmolarity and/or the controller preferably being programmed to obtain the value of osmolarity from a data storage.

4. The system of claim 1, wherein the controller is programmed to receive the overall osmolarity of the blood plasma as an input to the model of ultrafiltration, the system preferably comprising a user interface configured for inputting a value for the osmolarity and/or a measurement device for measuring osmolarity of a blood sample of the patient.

5. The system of claim 1, wherein the model uses a patient-specific parameter describing the effective hydraulic conductance and/or a patient-specific dissipation coefficient for crystalloid osmotic pressure to describe the membrane characteristics.

6. The system of claim 1, wherein the controller is programmed to fit the parameter values of the model at least on the basis of several consecutive values sensed over the course of a single dwell phase and/or sensed over different cycles of a treatment.

7. The system of claim 1, wherein the controller is programmed to determine, on the basis of several consecutive values sensed over the course of a treatment and/or a dwell phase, a curve of the intraperitoneal volume of the patient during the treatment and/or the dwell phase, which is compared to a corresponding curve determined by the model to fit the parameter values of the model, and/or wherein the controller is programmed to determine, on the basis of several consecutive values sensed over different cycles of a treatment, a curve of the intraperitoneal volume of the patient during the different cycles of the treatment, which is compared to a corresponding curve determined by the model to fit the parameter values of the model.

8. The system of claim 1, wherein the sensor is a pressure sensor configured to measure pressure values indicative of the intraperitoneal pressure of the patient, wherein preferably, the controller is programmed to determine, on the basis of the pressure values, the intraperitoneal volume of the patient, wherein preferably, the controller establishes a pressure/volume-characteristic of the individual patient in a first cycle and/or a fill and/or drain phase, and uses the pressure/volume-characteristic to determine the intraperitoneal volume during a dwell phase.

9. The system of claim 1, wherein the sensor is part of the peritoneal treatment machine and/or measures a pressure in an extracorporeal fluid line connected to the peritoneal treatment machine.

10. The system of claim 1, wherein the peritoneal treatment machine is configured to perform consecutive cycles differing in at least one out of fill volume, dwell time and composition of the dialysis solution, wherein the controller is programmed to use sensed values measured during the cycles for fitting the parameter values.

11. The system of claim 1, wherein the controller is programmed to output at least one parameter describing the current state of the peritoneum of the patient, and/or wherein the controller is programmed to accept, as an input, a planned prescription, and to output an ultrafiltration amount resulting from the planned prescription for the individual patient, the ultrafiltration amount calculated on the basis of the model.

12. The system of claim 1, wherein the controller is part of the peritoneal treatment machine, or wherein the controller is arranged remotely from the peritoneal treatment machine.

13. A system for predicting an amount of ultrafiltration of a peritoneal dialysis treatment for an individual patient, the system comprising:
a peritoneal treatment machine configured to perform cycles of a peritoneal treatment performed on the patient according to a prescription by controlling at least one actuator and/or valve of the peritoneal treatment machine, the cycles comprising a fill phase, a dwell phase and a drain phase,
a pressure sensor for repeatedly measuring pressure values indicative of the intraperitoneal pressure of the patient during the treatment performed on the patient,
a controller programmed to predict the magnitude of ultrafiltration volume expected during a treatment performed according to a prescription based on a model of ultrafiltration, and to fit parameter values of the model to the individual patient based on the values measured by the sensor,
wherein the controller is programmed:
to compare values calculated from the sensed pressure values with corresponding values calculated on the basis of the model for fitting the parameter values of the model to the individual patient and
to accept, as an input, a planned prescription, and to output an ultrafiltration amount resulting from the planned prescription for the individual patient, the ultrafiltration amount calculated on the basis of the model.

14. (canceled)

15. A method for predicting the magnitude of the ultrafiltration volume expected in a peritoneal dialysis treatment for an individual patient, the method comprising:
repeatedly measuring a sensed value during a peritoneal treatment performed by a peritoneal treatment machine according to a prescription,
predicting the magnitude of the ultrafiltration volume expected during a treatment performed according to a prescription based on a model of ultrafiltration, and
fitting parameter values of the model to the individual patient based on the values measured by the sensor,
wherein:
the model of ultrafiltration uses a patient-specific aggregated reflection coefficient as a parameter representing the overall average effect of different pores of the peritoneum of the individual patient and different solutes pre-sent in the peritoneal cavity and the blood plasma on the differential crystalloid osmotic pressure between the peritoneal cavity and blood plasma.
